(19) United States
(12) Patent Application Publication
(10) Pub. No.: US 2023/0112800 A1
(43) Pub. Date: Apr. 13, 2023

(54) DELAYED RELEASE NICLOSAMIDE FORMULATION

(71) Applicants: BOARD OF REGENTS, THE UNIVERSITY OF TEXAS SYSTEM, Austin, TX (US); TFF PHARMACEUTICALS, INC., Fort Worth, TX (US)

(72) Inventors: Robert O. WILLIAMS, III, Austin, TX (US); Zachary N. WARNKEN, Austin, TX (US); Miguel O. Jara GONZALES, Austin, TX (US); John J. KOLENG, Fort Worth, TX (US)

(73) Assignees: BOARD OF REGENTS, THE UNIVERSITY OF TEXAS SYSTEM, Austin, TX (US); TFF PHARMACEUTICALS, INC., Fort Worth, TX (US)

(21) Appl. No.: 17/823,635

(22) Filed: Aug. 31, 2022

Related U.S. Application Data
(60) Provisional application No. 63/239,333, filed on Aug. 31, 2021.

(51) Int. Cl. A61K 9/28; A61K 31/167; A61K 9/20

(52) U.S. Cl. CPC A61K 9/284; A61K 31/167; A61K 9/2866; A61K 9/2054; A61K 9/2059; A61K 9/2027; A61K 9/2013

(57) ABSTRACT

The present disclosure provides pharmaceutical compositions of niclosamide that may be administered orally. These compositions may allow the achievement of a therapeutically effective dose of niclosamide while preventing crystallization in the stomach which reduces bioavailability. These compositions may be used to treat one or more diseases or disorders such as a viral infection or cancer.

This application claims the benefit of priority to U.S. Provisional Application No. 63/239,333, filed on Aug. 31, 2021, the entire contents of which are hereby incorporated by reference.

This invention was made with government support under Grant No. R41 CA243931 awarded by the National Institutes of Health. The government has certain rights in the invention.

### BACKGROUND

### 1. Field

The present disclosure relates generally to the field of pharmaceuticals and pharmaceutical manufacture. More particularly, it concerns compositions and methods of preparing a pharmaceutical composition comprising niclosamide.

### 2. Description of Related Art

The WHO has declared the Coronavirus Disease 2019 (COVID-19) outbreak a pandemic (WHO Director-General's opening remarks at the Mission briefing on COVID-19—12 Mar. 2020; available on the world wide web at who.int/dg/speeches/detail/who-director-general-s-opening-remarks-at-the-mission-briefing-on-covid-19---12-march-2020). This virus is related to other coronaviruses that have created pandemics called the Severe Acute Respiratory Syndrome (SARS-CoV) in 2002 and the Middle East Respiratory Syndrome (MERS-CoV) in 2012 (Wu et al., 2004; Peeri et al., 2020). Currently, this COVID-19 has killed more people than the others two mentioned pandemics together (Gurwitz, 2020). This COVID-19 has been named as SARS-CoV-2 because its share near 80% of the genome with the SARS-CoV (Yan et al., 2020). Moreover, it has been reported that both viruses interact with similar affinity with angiotensin-converting enzyme 2 (ACE2), a protein that works as an entry receptor (Ahmed et al., 2020; Walls et al., 2020).

Unfortunately, there are no specific drugs for coronaviruses (Walls et al., 2020; Tortoric et al., 2019). The present strategy in drug discovery has been the test of drugs previously used in SARS and MERS (Wang et al., 2020). Recently, the drug chloroquine was successful against an in-vitro isolate COVID-19 (Vero E6 cells) with an IC₅₀ of 1.13 μM. The mechanism was attributed to an increased endosomal pH, one of the same mechanisms reported for another drug called niclosamide (Jurgeit et al., 2012; Vincent et al., 2005; Wang et al., 2018). In Vero cells infected by SARS-CoV (2002 pandemic), the reported IC₅₀ of chloroquine is 4.4 μM, yet niclosamide inhibits viral replication with an IC₅₀<0.1 μM (Wen et al., 2007). In a separate experiment, SARS-CoV replication was completely inhibited using niclosamide at concentrations between 1.56-3.12 μM (Wu et al., 2004). For these reasons, niclosamide has been proposed as a candidate for this COVID-19 pandemic and has recently shown an IC₅₀ of 0.28 μM (Xu et al., 2020; Jeon et al., 2020).

Niclosamide has been used for 60 years, it is an FDA approved anthelmintic drug that is listed as an Essential Medicine by the WHO (Barbosa et al., 2019). Niclosamide has been proposed as a candidate for repurposing as a multi-targeted cancer therapy, broad-spectrum antiviral, and antibacterial, among several others (Xu et al., 2020; Li et al., 2014; Chen et al., 2018; Tam et al., 2018). It seems that the main feature that allows all those effects are the physical chemistry of the molecule itself instead of specific ligand-receptor interactions (Fonseca et al., 2012). Niclosamide is well known for its protonophoric activity, in other words, the capability of transporting protons through membranes and disrupting pH gradients that regulate several key signaling pathways (Jurgeit et al., 2012; Xu et al., 2020; Li et al., 2014; Chen et al., 2018; Tam et al., 2018; Fonseca et al., 2012; Circu et al., 2016; Ippolito et al., 2016; Mook et al., 2015; Tharmalingam et al., 2018). In CoVs, niclosamide has inhibited MERS-CoV replication more than 1000-fold by modifying pathways related to the proteasome and autophagy mechanisms (Xu et al., 2020; Gassen et al., 2019). This feature can make niclosamide a host-directed broad-spectrum antiviral (Chen et al., 2018). The main limitation of these studies is that they were conducted using DMSO as a solvent.

Niclosamide has been effective in SARS-CoV (Wu et al., 2014; Wen et al., 2007) and MERS-CoV (Wu et al., 2014; Wen et al., 2007; Gassen et al., 2019). It has been proposed that niclosamide can be a therapeutic option for SARS-CoV-2 (Xu et al., 2020). As stated earlier, SARS-Cov-2 targets ACE2 which is not only expressed in the lungs (main entry) but also in intestine, kidney, and blood vessels (Fang et al., 2020). This increases the risk in populations with diabetes and hypertension that normally upregulate those receptors (Fang et al., 2020). Some patients undergo gastrointestinal symptoms similar to SARS-CoV and MERS-CoV and cardiac problems (Rothan & Byrareddy, 2020). SARS-CoV can replicated in the intestinal lumen and niclosamide orally could be helpful.

While oral compositions have been formulated, these compositions crystallized in acidic environments such as the stomach destroying the potential solubility improvements seen in the amorphous forms.

### SUMMARY

The present disclosure provides pharmaceutical compositions comprising delayed release compositions of niclosamide for administration orally. Thus, compositions of the invention allow targeting and delivery of the ASD to the intestines and not the acidic environment of the stomach where niclosamide from the ASD will crystallize. Without wishing to be bound by any theory, these compositions may have one or more advantageous properties such as higher drug loading, maintenance of a therapeutically effective dose, or other properties such as ability to more effectively deliver the drug to the target organ.

In some aspects, the present disclosure provides delayed release niclosamide compositions comprising:

(A) a core comprising:
(i) niclosamide;
(ii) a pharmaceutically acceptable polymer;
(iii) an amorphous solid dispersion (ASD) excipient;
wherein the niclosamide, the pharmaceutically acceptable polymer, and the ASD excipient form an amorphous solid dispersion; and
(iv) a first core excipient; and
   (B) a delayed release coating comprising a delayed release polymer;
   wherein the core is encapsulated within the delayed release coating.

In some embodiments, the composition is a tablet. In some embodiments, the delayed release coating encapsulates the core until the delayed release coating is exposed to a pH of greater than 7.4. In some embodiments, the delayed release coating substantially encapsulates the core. In some embodiments, the delayed release coating essentially encapsulates the core. In some embodiments, the delayed release coating entirely encapsulates the core.

In some embodiments, the pharmaceutically acceptable polymer is a vinylpyrrolidone copolymer. In some embodiments, the pharmaceutically acceptable polymer is a 1-vinyl-2-pyrrolidone and vinyl acetate copolymer. In some embodiments, the pharmaceutically acceptable polymer is copovidone. In some embodiments, the composition further comprises a sealing coating. In some embodiments, the sealing coating is a polyvinyl alcohol or a cellulose polymer. In some embodiments, the sealing coating is polyvinyl alcohol.

In some embodiments, the delayed release coating is a cellulose polymer or an acrylate copolymer. In some embodiments, the delayed release coating is a cellulose polymer. In some embodiments, the delayed release coating is ethyl cellulose, hypromellose phthalate, or hypromellose acetate succinate.

In some embodiments, the sealing coating encapsulates the core. In some embodiments, the sealing coating substantially encapsulates the core. In some embodiments, the sealing coating essentially encapsulates the core. In some embodiments, the sealing coating entirely encapsulates the core. In some embodiments, the delayed release coating encapsulates the sealing coating. In some embodiments, the delayed release coating substantially encapsulates the sealing coating. In some embodiments, the delayed release coating essentially encapsulates the sealing coating. In some embodiments, the delayed release coating entirely encapsulates the sealing coating.

In some embodiments, the delayed release coating is an acrylate copolymer such as a copolymer of methacrylate and acrylic acid. In some embodiments, the delayed release coating is a copolymer of methacrylate and ethyl acrylate. In some embodiments, the acrylate copolymer is a copolymer of methacrylate and ethyl acrylate comprising a ratio of methacrylate units and acrylic acid units is from 10:1 to about 1:10. In some embodiments, the ratio is from about 5:1 to 1:5 such as about 2:1 to about 1:2. In some embodiments, the ratio is about 1:1.

In some embodiments, the ASD excipient is a vitamin or vitamin derivative such as a vitamin derivative. In some embodiments, the derivative is a pegylated version of the vitamin. In some embodiments, the vitamin derivative is a vitamin E derivative. In some embodiments, the vitamin derivative is D-α-tocopheryl polyethylene glycol succinate.

In some embodiments, the first core excipient is cellulose such as cellulose having a particle size from about 1 μm to about 500 μm. In some embodiments, the particle size is from about 10 μm to about 300 μm. In some embodiments, the particle size is from about 15 μm to about 200 μm. In some embodiments, the cellulose has a bulk density is from about 0.05 g/cm³ to about 1.0 g/cm³. In some embodiments, the bulk density is from about 0.1 g/cm³ to about 0.75 g/cm³. In some embodiments, the bulk density is from about 0.2 g/cm³ to about 0.5 g/cm³.

In some embodiments, the core further comprises a second core excipient. In some embodiments, the second core excipient is a modified cellulose derivative. In some embodiments, the modified cellulose derivative is carboxymethyl cellulose. In some embodiments, the modified cellulose derivative is a cross linked carboxymethyl cellulose. In some embodiments, the modified cellulose derivative has a degree of substitution from about 0.4 to about 1.2. In some embodiments, the degree of substitution from about 0.5 to about 1.0. In some embodiments, the degree of substitution from about 0.55 to about 0.9. In some embodiments, the modified cellulose derivative is a monovalent metal salt such as a sodium salt.

In some embodiments, the second core excipient is a modified starch derivative. In some embodiments, the modified starch derivative comprises one or more carboxymethyl or a salt thereof. In some embodiments, the modified starch derivative comprises a salt of a carboxymethyl. In some embodiments, the salt is an alkali metal salt such as a sodium salt. In some embodiments, the modified starch derivative is sodium starch glycolate. In some embodiments, the second core excipient is a vinylpyrrolidone copolymer. In some embodiments, the vinylpyrrolidone copolymer is a 1-vinyl-2-pyrrolidone and vinyl acetate copolymer. In some embodiments, the vinylpyrrolidone copolymer is copovidone.

In some embodiments, the core further comprises a third core excipient. In some embodiments, the third core excipient is a salt such as a sodium salt. In some embodiments, the salt is sodium chloride or sodium bicarbonate.

In some embodiments, the core further comprises a fourth core excipient. In some embodiments, the fourth core excipient is a lubricant. In some embodiments, the lubricant is a hydrophobic lubricant. In other embodiments, the lubricant is a hydrophilic lubricant. In some embodiments, the lubricant is a fatty acid, a salt of a fatty acid, sodium lauryl sulfate, magnesium stearate, magnesium silicate, calcium stearate, sodium lauryl sulphate, sodium stearyl fumarate, magnesium lauryl sulphate, stearic acid, calcium stearate, glyceryl behenate, behenoyl polyoxylglycerides, glyceryl dibehenate, lauric acid, glyceryl monostearate, glyceryl tristearate, myristic acid, palmitic acid, poloxamer, polyethylene glycol, polyethylene glycol 3350, polyoxyl 10 oleyl ether, polyoxyl 15 hydroxystearate, polysorbate 40, polyoxyl 20 cetostearyl ether, polyoxyl 40 stereate, potassium benzoate, sodium benzoate, sorbitan monolaurate, sorbitan monooleate, sodium stearate, sorbitan monopalmitate, sorbitan monostearate, zinc stearate, sorbitan sesquioleate, sorbitan trioleate, or talc. In some embodiments, the fourth core excipient is a fatty acid or a salt of a fatty acid. In some embodiments, the fourth core excipient is a salt of a fatty acid. In some embodiments, the fourth core excipient is a derivative of stearic acid such as a salt of stearic acid. In some embodiments, the fourth core excipient is magnesium stearate.

In some embodiments, the core further comprises a fifth core excipient. In some embodiments, the fifth core excipient is a filler. In some embodiments, the fifth core excipient is talc or silicone dioxide such as colloidal silicone dioxide.

In some embodiments, the delayed release coating further comprises a first delayed release coating excipient. In some embodiments, the first delayed release coating excipient is a plasticizer such as citric acid or an ester of citric acid. In some embodiments, the plasticizer is an ester of citric acid such as triethyl citrate.

In some embodiments, the delayed release coating further comprises a second delayed release coating excipient. In some embodiments, the second delayed release coating excipient is an anti-foaming agent such as a siloxane. In some embodiments, the anti-foaming agent further comprises silicon dioxide. In some embodiments, the anti-foaming agent is a mixture of a siloxane and silicon dioxide. In some embodiments, the anti-foaming agent is simethicone.

In some embodiments, the composition comprises an amount of niclosamide from about 5% to about 40% by weight of the total composition. In some embodiments, the amount of niclosamide is from about 10% to about 30% by weight of the total composition. In some embodiments, the amount of niclosamide is from about 15% to about 25% by weight of the total composition. In some embodiments, the composition comprises an amount of the pharmaceutically acceptable polymer from 10% to about 60% by weight of the total composition. In some embodiments, the amount of the pharmaceutically acceptable polymer is from about 20% to about 50% by weight of the total composition. In some embodiments, the amount of the pharmaceutically acceptable polymer from about 30% to about 40% by weight of the total composition. In some embodiments, the composition comprises an amount of the ASD excipient is from about 0.5% to about 10% by weight of the total composition. In some embodiments, the amount of the ASD excipient is from about 1% to about 5% by weight of the total composition. In some embodiments, the amount of the ASD excipient is from about 2% to about 4% by weight of the total composition.

In some embodiments, the composition comprises an amount of a first core excipient from about 5% to about 30% by weight of the total composition. In some embodiments, the amount of the first core excipient is from about 10% to about 25% by weight of the total composition. In some embodiments, the amount of the first core excipient is from about 10% to about 20% by weight of the total composition. In some embodiments, the composition comprises an amount of a second core excipient from about 1% to about 20% by weight of the total composition. In some embodiments, the amount of the second core excipient is from about 2.5% to about 15% by weight of the total composition. In some embodiments, the amount of the second core excipient is from about 5% to about 10% by weight of the total composition. In some embodiments, the composition comprises an amount of a third core excipient from about 2.5% to about 20% by weight of the total composition. In some embodiments, the amount of the third core excipient is from about 5% to about 15% by weight of the total composition. In some embodiments, the amount of the third core excipient is from about 8% to about 12% by weight of the total composition. In some embodiments, the composition comprises an amount of a fourth core excipient from about 0.05% to about 1% by weight of the total composition. In some embodiments, the amount of the fourth core excipient is from about 0.1% to about 0.5% by weight of the total composition. In some embodiments, the amount of the fourth core excipient is from about 0.2% to about 0.3% by weight of the total composition.

In some embodiments, the composition comprises an amount of the sealing coating from about 0.1% to about 10% by weight of the total composition. In some embodiments, the amount of the sealing coating is from about 0.5% to about 5.0% by weight of the total composition.

In some embodiments, the amount of the sealing coating is from about 1.0% to about 3.0% by weight of the total composition.

In some embodiments, the composition comprises an amount of the delayed release coating is from about 0.5% to about 15% by weight of the total composition. In some embodiments, the amount of the delayed release coating is from about 1.0% to about 10.0% by weight of the total composition. In some embodiments, the amount of the delayed release coating is from about 2.5% to about 7.5% by weight of the total composition. In some embodiments, the composition comprises an amount of the first delayed release coating excipient is from about 0.05% to about 2.5% by weight of the total composition. In some embodiments, the amount of the first delayed release coating excipient is from about 0.1% to about 1.5% by weight of the total composition. In some embodiments, the amount of the first delayed release coating excipient is from about 0.25% to about 1.0% by weight of the total composition. In some embodiments, the composition comprises an amount of the second delayed release coating excipient is from about 0.001% to about 0.1% by weight of the total composition. In some embodiments, the amount of the second delayed release coating excipient is from about 0.0025% to about 0.075% by weight of the total composition. In some embodiments, the amount of the second delayed release coating excipient is from about 0.0025% to about 0.025% by weight of the total composition.

In some embodiments, at least 90% of the niclosamide and the pharmaceutically acceptable polymer is present in an amorphous form. In some embodiments, at least 95% of the niclosamide and the pharmaceutically acceptable polymer is present in an amorphous form. In some embodiments, at least 98% of the niclosamide and the pharmaceutically acceptable polymer is present in an amorphous form. In some embodiments, at least 99% of the niclosamide and the pharmaceutically acceptable polymer is present in an amorphous form.

In some embodiments, less than 10% of the niclosamide is present in the crystalline phase after entry into the small intestine as measured by a pH transition dissolution test (acid-base). In some embodiments, less than 5% of the niclosamide is present in the crystalline phase after entry into the small intestine. In some embodiments, less than 3% of the niclosamide is present in the crystalline phase after entry into the small intestine.

In some embodiments, the sealing coating further comprises a sealing coating excipient. In some embodiments, the sealing coating excipient is titanium oxide. In other embodiments, the sealing coating excipient is a polyethylene glycol. In some embodiments, the polyethylene glycol comprises an average molecular weight from about 1000 daltons to about 10,000 daltons. In some embodiments, the average molecular weight is from about 2,000 daltons to about 5,000 daltons. In some embodiments, the average molecular weight is from about 3,000 daltons to about 4,000 daltons.

In some embodiments, the composition comprises:
(A) a core comprising:

(i) niclosamide;

(ii) copovidone;

(iii) an ASD excipient; wherein the ASD excipient is a pegylated version of Vitamin E;

wherein the niclosamide, the copovidone, and the ASD excipient form an amorphous solid dispersion;

(iv) a first core excipient, wherein the first core excipient is cellulose;

(v) a second core excipient, wherein the second core excipient is cross linked carboxymethyl cellulose;

(vi) a third core excipient, wherein the third core excipient is a salt;

(vii) a fourth core excipient, wherein the fourth core excipient is a fatty acid or salt thereof;
(B) a sealing coating;

wherein the sealing coating comprises a polyvinyl alcohol polymer; and
(C) a delayed release coating;

wherein the delayed release coating comprises an acrylate copolymer, a first delayed release coating excipient, and a second delayed release coating excipient, wherein;

(i) the first delayed release coating excipient is triethyl citrate;

(ii) the second delayed release coating excipient is simethicone.

In still another aspect, the present disclosure provides methods of preparing a composition described herein comprising:
(A) preparing an extrudate comprising niclosamide and a pharmaceutically acceptable polymer to obtain an amorphous solid dispersion;
(B) admixing one or more core excipients to the amorphous solid dispersion to obtain a precursor core;
(C) pressing the precursor core to obtain a core;
(D) coating the core with a clear coating to obtain a clear coated core; and
(E) coating the clear coated core with an acrylate copolymer to obtain a composition.

In some embodiments, the extrudate is prepared using a hot melt extruder. In some embodiments, the hot melt extrusion is conducted at a temperature from about 100° C. to about 240° C. In some embodiments, the amorphous solid dispersion further comprises an excipient. In some embodiments, the extrudate further comprises a second core excipient. In some embodiments, the extrudate further comprises a third core excipient. In some embodiments, the extrudate further comprises a fourth core excipient. In some embodiments, the second core excipient, third core excipient, fourth excipient, or fifth core excipient is admixed with the amorphous solid dispersion. In some embodiments, the second core excipient, third core excipient, fourth excipient, and fifth core excipient is admixed with the amorphous solid dispersion.

In some embodiments, the core excipients are admixed with the amorphous solid dispersion for a time period from about 3 minutes to about 60 minutes. In some embodiments, the time period is from about 5 minutes to about 30 minutes. In some embodiments, the time period is from about 8 minutes to about 15 minutes. In some embodiments, the core excipients are admixed with the amorphous solid dispersion at a blending rate from about 5 rpm to about 100 rpm. In some embodiments, the blending rate is from about 10 rpm to about 50 rpm. In some embodiments, the blending rate is from about 20 rpm to about 30 rpm. In some embodiments, the admixing comprises sieving the core excipients together.

In some embodiments, the precursor core is pressed into a core using a rotary tablet press. In some embodiments, the clear coating is added with a target weight gain of at least 1%. In some embodiments, the clear coating is added with a target weight gain of at least 2%. In some embodiments, the clear coating is added with a target weight gain of at least 3%. In some embodiments, the acrylate copolymer is added with a target weight gain of at least 2.5%. In some embodiments, the acrylate copolymer is added with a target weight gain of at least 5%. In some embodiments, the acrylate copolymer is added with a target weight gain of at least 7.5%. In some embodiments, the acrylate copolymer is admixed with one or more delayed release coating excipients prior to coating.

In some embodiments, the acrylate copolymer is admixed with a first delayed release coating excipient and a second delayed release coating excipient prior to coating. In some embodiments, the acrylate copolymer, the first delayed release coating excipient, and the second delayed release coating excipient are admixed into a mixture prior to coating. In some embodiments, the mixture is sieved before coating. In some embodiments, the sieve is 60 mesh.

In still yet another aspect, the present disclosure provides pharmaceutical compositions prepared according to the method described herein

In another aspect, the present disclosure provides methods of treating or preventing a disease or disorder comprising administering to a patient a therapeutically effective amount of the form of niclosamide described herein or the pharmaceutical composition described herein. In some embodiments, the method treats the disease or disorder. In other embodiments, the method prevents the disease or disorder. In some embodiments, the disease or disorder is treatable by the administration of niclosamide. In other embodiments, the disease or disorder is preventable by the administration of niclosamide. In some embodiments, the methods comprise administering niclosamide once. In other embodiments, the methods comprise administering niclosamide two or more times. In some embodiments, the disease or disorder is an infectious disease or a cancer. In some embodiments, the infectious disease is a viral infection, a bacterial infection, or a helminth infection. In some embodiments, the cancer is castration-resistant prostate cancer, colorectal cancer, glioblastoma, or lung cancer.

In yet another aspect, the present disclosure provides dosage forms of niclosamide described herein or the pharmaceutical composition described herein for use in the treatment or prevention of a disease or disorder. In some embodiments, the disease or disorder is an infectious disease or a cancer. In some embodiments, the infectious disease is a viral infection, a bacterial infection, or a helminth infection. In some embodiments, the cancer is castration-resistant prostate cancer, colorectal cancer, glioblastoma, or lung cancer.

In still yet another aspect, the present disclosure provides uses of the dosage form of niclosamide described herein, the pharmaceutical composition described herein, or the method described herein in the manufacture of a medicament for the treatment or prevention of a disease or disorder. In some embodiments, the disease or disorder is an infectious disease or a cancer. In some embodiments, the infectious disease is a viral infection, a bacterial infection, or a helminth infection. In some embodiments, the cancer is castration-resistant prostate cancer, colorectal cancer, glioblastoma, or lung cancer.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the dissolution test of Niclosamide 40%-VA64® 60% extrudate (amorphous/glass) and physical mixture (PM).

FIG. 2 shows the dissolution test of Niclosamide 35%-VA64® 60%-TPGS 5% extrudate.

FIG. 3 shows the dissolution test of Niclosamide-VA64® extrudates at different drug loadings (DL).

FIG. 4 shows the dissolution test of Niclosamide 20%-VA64® 75%-5% surfactant/emulsifiers.

FIG. 5 shows the dissolution test of Niclosamide-Soluplus® (Sol) extrudates at different drug loadings (DL).

FIG. 6 shows the dissolution test of Niclosamide-Kollidon 30® (polyvinylpyrrolidone) at 40 and 50% drug loading (DL).

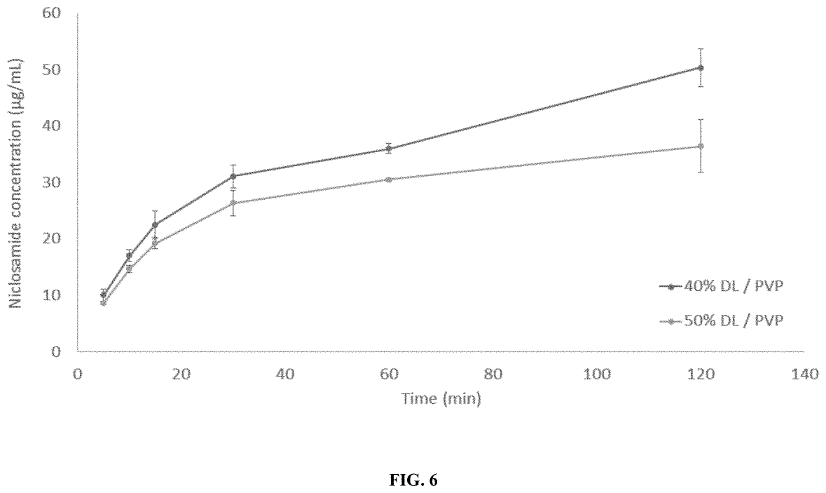

FIG. 7 shows the niclosamide plasma concentration as a function of time in mice dosed with the niclosamide amorphous solid dispersion compared to crystalline niclosamide.

FIG. 8 shows the dissolution profile of niclosamide ASD, its physical mixture (PM), and niclosamide anhydrate in FaSSIF media. The samples were taken and passed through 0.2 μm filters. The particle size distribution of niclosamide ASD after 2 h in FaSSIF and a FaSSIF control.

FIG. 9 shows the diffusion profiles of niclosamide ASD and niclosamide anhydrate. The donor and receiver cells were filled with FaSSIF and decanol, respectively.

FIG. 10 shows the pH-shift dissolution test of niclosamide ASD.

FIG. 11 shows the pharmacokinetic profiles (in rats) of niclosamide anhydrate suspended in FaSSIF, niclosamide ASD suspended in FaSSIF, and niclosamide ASD in capsules (n=5).

FIG. 12 shows the dissolution profile for 100 mg niclosamide delayed-release tablets at 10% wg prepared in Example 2A using the method for measurement described in Example 2C (These tablets were stored at room temperature with desiccant bags for 6 months, n=3).

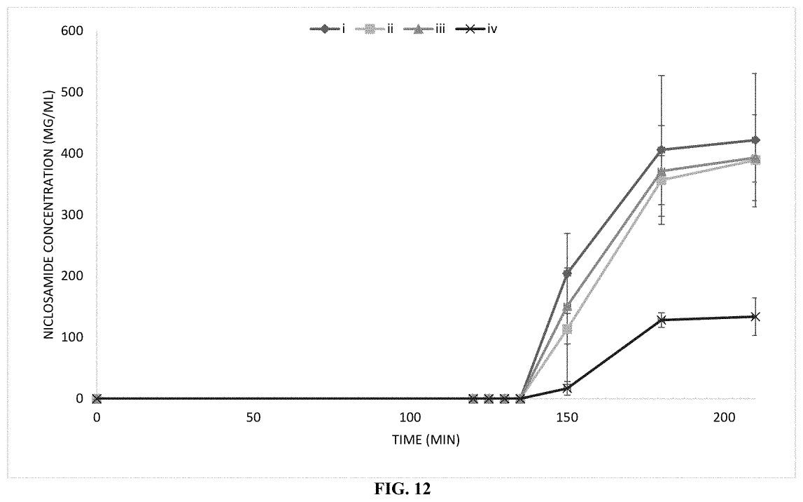

FIG. 13 shows the dissolution profile for 100 mg niclosamide delayed-release tablets at 7.5% wg prepared in Example 2A using the method for measurement described in Example 2C (These tablets were stored at room temperature with desiccant bags for 6 months, n=3).

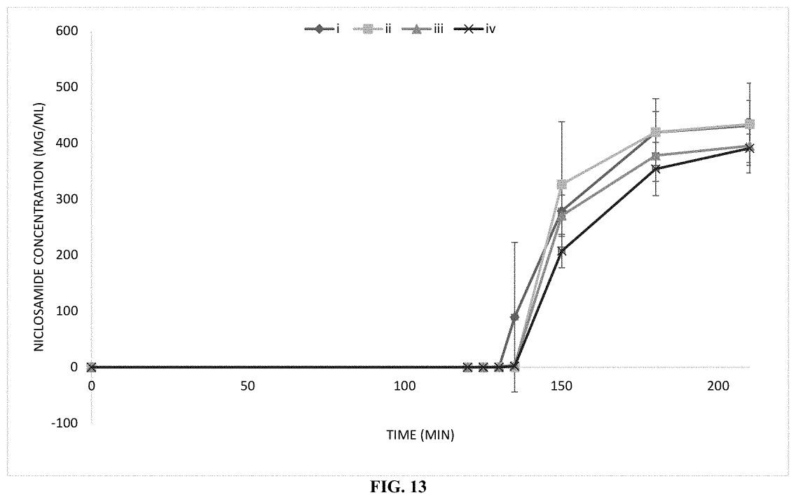

FIG. 14 shows the dissolution profile for 100 mg niclosamide delayed-release tablets at 5% wg prepared in Example 2A using the method for measurement described in Example 2C (These tablets were stored at room temperature with desiccant bags for 6 months, n=3).

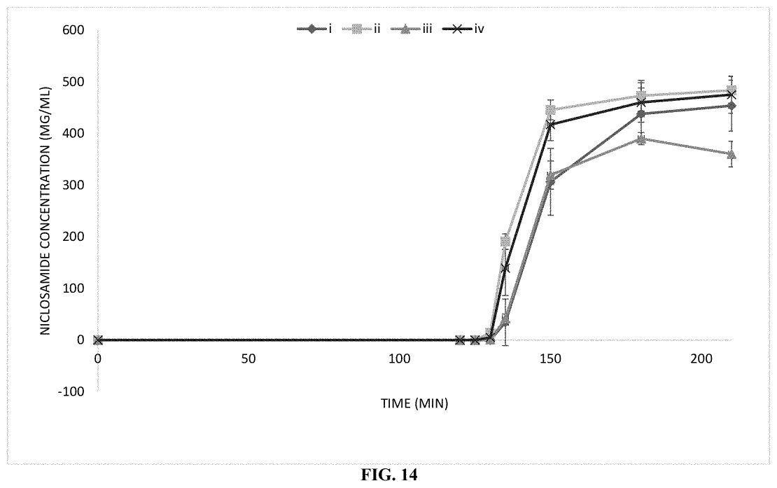

FIG. 15 shows the dissolution profile of unencapsulated granules of niclosamide ASD (280 mg) and encapsulated granules of niclosamide ASD contained in enteric capsules size 0 (400 mg of 25% Sodium bicarbonate, 5% Explotab®, and 70% ASD) prepared in I using the method for measurement described in I (n=3).

FIG. 16 shows the dissolution profile for encapsulated granules of niclosamide ASD after being exposed for 2 hours in acidic medium followed by introduction of a model medium for intestinal fluid.

### DETAILED DESCRIPTION

In some aspects of the present disclosure, the pharmaceutical compositions provided herein may comprise niclosamide in formulations for oral administration that result in delay release of the niclosamide. In some aspects, the present compositions may be used to deliver a therapeutically effective dose to the intestines without the niclosamide crystallizing in the stomach. The present disclosure also provides methods of preparing these compositions and uses of these compositions to treat a disease or disorder such as a microbial infection or cancer. A detailed description of these compositions is provided below.

### I. Pharmaceutical Compositions and Methods of Use

In some aspects, the present disclosure provides delayed release pharmaceutical compositions containing an active agent, such as niclosamide, and may optionally contain an excipient. Theses composition may be formulated as tablets for administration orally.

Niclosamide is a poorly water soluble, lipophilic molecule previously known to have poor and variable bioavailability which for its current approved indication for treating helminthic infections in the gastrointestinal tract is not a limiting factor. When attempting to repurpose the medication for the treatment of diseases such as prostate cancer which require systemic concentrations of the drug, the challenges to overcome the bioavailability limitations become clear. As niclosamide is both poorly water soluble and lipophilic, the rate limiting step for the oral absorption of the drug is the dissolution of the molecule.

Amorphous solid dispersions are used to improve the solubility and bioavailability of poorly water-soluble drugs. They are used to overcome limitations of solubility by the pharmaceutical industry in 19 commercial products approved by the Food & Drug Administration between 2007 and 2017. Most often these products are based on binary mixtures of the drug and a hydrophilic polymer. However, these formulations can be limited for drugs with specific physicochemical properties and dose requirements, such as highly lipophilic drugs like atovaquone (Friesen et al., 2008).

The delayed release compositions of the present disclosure comprise a core and a delayed release coating. In some aspects, the compositions further comprise a sealing coating positioned between the core and the delayed release coating. In some embodiments, the core comprises niclosamide, a pharmaceutically acceptable polymer, an amorphous solid dispersion (ASD) excipient, and a first core excipient, wherein the niclosamide, the pharmaceutically acceptable polymer, and the ASD excipient together form an amorphous solid dispersion. In some aspects, the core further comprises a second core excipient, a third core excipient, a fourth core excipient, and/or a fifth core excipient.

The present disclosure provides methods of treating a patient with delayed release niclosamide formulations. Several clinical indications would benefit from administration of niclosamide compositions with enhanced bioavailability. These indications include infections of a microorganism such as bacteria, a virus, a parasite, or a helminth (e.g., a tapeworm). In particular, the compositions may be used to treat a viral infection. Some non-limiting examples of viral infections which may be treated with the composition described herein include COVID-19, MERS, SARS, influenza, Zika, Lassa, Ebola, HIV including HIV with complications such as TB, and adenovirus. With regards to viral infections, some viruses such as SARS-CoV can enter cells and replicate where ACE2⁺ tissues are present, which includes areas such as the kidneys, lungs, and small intestine (Hoffmann et al., 2020). In other embodiments, the pharmaceutical compositions may be used to treat schistosomiasis and related pulmonary complications. Additionally, these pharmaceutical compositions may be used to treat vancomycin resistant enterococci, Pseudomonas aeruginosa, Acinetobacter baumannii, Klebsiella pneumoniae, C. difficile, or MRSA. Furthermore, the pharmaceutical composition may be used to treat or control diabetes. Other such clinical indications include several cancers, in particular prostate cancer (e.g., castration-resistant prostate cancer), colorectal cancer, glioblastoma, breast cancer, or lung cancer.

In some embodiments, the pharmaceutical composition may be administered on a routine schedule. As used herein, a routine schedule refers to a predetermined designated period of time. The routine schedule may encompass periods of time, which are identical or which differ in length, as long as the schedule is predetermined. For instance, the routine schedule may involve administration four times a day, three times a day, twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In some embodiments, niclosamide is administered once per day. In preferred embodiments, niclosamide is administered less than four times per day. In some embodiments, a complete dose of niclosamide is between from about 100 mg to about 5 g, such as 100 mg, 200 mg, 300 mg, 400 mg, 500 mg, 600 mg, 700 mg, 800 mg, 900 mg, 1 g, 1.25 g, 1.5 g, 1.75 g, 2 g, 2.5 g, 3 g, 3.5 g, 4 g, 4.5 g, to about 5 g, or any range derivable therein.

In some embodiments, the pharmaceutical composition may be used to treat one or more diseases or disorders in combination with one or more additional active agents. In particular, the pharmaceutical composition may be used in conjunction with another antimicrobial agent or active agent which reduces one or more symptoms of the microbial infection. Some non-limiting examples of additional therapeutic agents may include chloroquine, hydroxychloroquine, thalidomide, plasminogen, colistin, polymyxin B, or clofazimine. In other compositions, the pharmaceutical composition may be used in conjunction with one or more anti-cancer agents such as a chemotherapeutic agent, radiotherapy, surgery, or immunotherapy. Some non-limiting examples of additional therapeutic agents may include abiraterone such as abiraterone acetate, enzalutamide, or bicalutamide.
A. Core

In some embodiments, the core comprises niclosamide, a pharmaceutically acceptable polymer, an amorphous solid dispersion (ASD) excipient, and a first core excipient, wherein the niclosamide, the pharmaceutically acceptable polymer, and the ASD excipient together form an amorphous solid dispersion. In some aspects, the core further comprises a second core excipient, a third core excipient, a fourth core excipient, and/or a fifth core excipient.

1. Niclosamide

The compositions described herein comprise niclosamide as an active agent. The tablets described herein contain niclosamide in an amount between about 5% to about 40% w/w, between about 10% to about 30% w/w, between about 15% to about 25% w/w of the total composition. In some embodiments, the amount of the niclosamide is from about 2.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 32.5%, 35%, 37.5%, to about 40% w/w or any range derivable therein.

In some aspects, a wide variety of different forms of niclosamide may be used. Niclosamide is an active agent with a chemical name of 5-Chloro-N-(2-chloro nitrophenyl)salicylamide. The niclosamide used herein may be either anhydrous or may be a hydrate of niclosamide such as monohydrate of niclosamide. Furthermore, the niclosamide may be a salt, such as an ethanolamine or piperazine salt. Additionally, co-crystal of niclosamide may be used in the pharmaceutical compositions, which may include co-crystals of niclosamide with 2-aminothiazole, benzamide, isoniazid, acetamide, caffeine, urea, p-aminobenzoic acid, theophylline, nicotinamide, or isonicotinamide (Sanphui et al., 2012; Luedeker et al., 2016). Alternative, it is also contemplated that known derivatives, such as those described by Mook et al., 2015, which is incorporated herein by reference, may also be used in the formulations.

Additionally, niclosamide is light sensitive and should be stored in the dark to protect the composition from light.

2. Pharmaceutically Acceptable Polymers

In some aspects, the present disclosure provides compositions which may further comprise a pharmaceutically acceptable polymer. In some embodiments, the polymer has been approved for use in a pharmaceutical formulation and is known to undergo softening or increased pliability when raised above a specific temperature without substantially degrading, i.e., less than about 10%, less than about 5%, less than about 2%, or less than about 1% of the polymer degrades when raised above the specific temperature.

When a pharmaceutically acceptable polymer is present in the composition, the pharmaceutically acceptable polymer is present in the composition in an amount between 10% to 60% w/w, between 20% to 50% w/w, or between 30% to 40% w/w. In some embodiments, the amount of the pharmaceutically acceptable polymer is from about 5%, 10%, 15%, 50%, 20%, 25%, 30%, 31%, 32%, 32.5%, 33%, 34%, 35%, 36%, 37%, 37.5%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 65%, 70%, 80%, to about 90% w/w or any range derivable therein.

Within the compositions described herein, a single polymer or a combination of multiple polymers may be used. In some embodiments, the polymers used herein may fall within two classes: cellulosic and non-cellulosic. These classes may be further defined by their respective charge into neutral and ionizable. Ionizable polymers have been functionalized with one or more groups which are charged at a physiologically relevant pH. Some non-limiting examples of neutral non-cellulosic polymers include polyvinyl pyrrolidone, polyvinyl alcohol, copovidone, and poloxamer. Within this class, in some embodiments, pyrrolidone containing polymers are particularly useful. Some non-limiting examples of ionizable cellulosic polymers include cellulose acetate phthalate and hydroxypropyl methyl cellulose acetate succinate. Finally, some non-limiting examples of neutral cellulosic polymers include hydroxypropyl cellulose, hydroxypropyl methyl cellulose, hydroxyethyl cellulose, and hydroxymethyl cellulose.

Some specific pharmaceutically acceptable polymers which may be used include, for example, Eudragit™ RS PO, Eudragit™ S100, Kollidon SR (poly(vinyl acetate)-co-poly(vinylpyrrolidone) copolymer), Ethocel™ (ethylcellulose), HPC (hydroxypropylcellulose), cellulose acetate butyrate, poly(vinylpyrrolidone) (PVP), poly(ethylene glycol) (PEG), poly(ethylene oxide) (PEO), poly(vinyl alcohol) (PVA), hydroxypropyl methylcellulose (HPMC), ethylcellulose (EC), hydroxyethylcellulose (HEC), carboxymethyl cellulose and alkali metal salts thereof, such as sodium salts sodium carboxymethyl-cellulose (CMC), dimethylaminoethyl methacrylate-methacrylic acid ester copolymer, carboxymethylethyl cellulose, carboxymethyl cellulose butyrate, carboxymethyl cellulose propionate, carboxymethyl cellulose acetate butyrate, carboxymethyl cellulose acetate propionateethylacrylate-methylmethacrylate copolymer (GA-MMA), C-5 or 60 SH-50 (Shin-Etsu Chemical Corp.), cellulose acetate phthalate (CAP), cellulose acetate trimelletate (CAT), poly(vinyl acetate) phthalate (PVAP), hydroxypropylmethylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, poly(methacylic acid-co-methyl methacrylate 1:2), poly(methacrylic acid-co-methyl methacrylate 1:1), Poly(methyl acrylate -co-methyl methacrylate-co-methacrylic acid 7:3:1), poly(butyl methacrylate-co-(2-dimethylaminoethyl) methacrylate-co-methyl methacrylate 1:2:1), poly(ethyl acrylate-co-methyl methacrylate 2:1), poly(ethyl acrylate-co-methyl methacrylate 2:1), poly(ethyl acrylate-co-methyl methacrylate-co-trimethylammonioethyl methacrylate chloride 1:2:0.2), poly(ethyl acrylate -co-methyl methacrylate-co-trimethylammonioethyl methacrylate chloride 1:2:0.1), Eudragit L-30-D™ (MA-EA, 1:1), Eudragit L-100-55™ (MA-EA, 1:1), hydroxypropylmethylcellulose acetate succinate (HPMCAS), polyvinyl caprolactam-polyvinyl acetate-PEG graft copolymer, polyvinyl alcohol/acrylic acid/methyl methacrylate copolymer, polyalkylene oxide, Coateric™ (PVAP), Aquateric™ (CAP), and AQUACOAT™ (HPMCAS), polycaprolactone, starches, pectins, chitosan or chitin and copolymers and mixtures thereof, and polysaccharides such as tragacanth, gum arabic, guar gum, and xanthan gum.

In some embodiments, the compositions described herein contain a pharmaceutically acceptable polymer selected from povidone, copovidone, polyvinyl pyrrolidone, polyvinyl acetate, and SOLUPLUS® (polyvinyl caprolactampolyvinyl acetate-polyethylene glycol graft co-polymer, commercially available from BASF). In particular, the pharmaceutical acceptable polymer may be a copolymer of polyvinyl pyrrolidone and polyvinyl acetate. In particular, the copolymer may comprise about 5-7 vinyl pyrrolidone units to about 3-5 units of vinyl acetate, in particular 6 units of vinyl pyrrolidone and 4 units of vinyl acetate. The number-average of the molecular weight of the polymer may be from about 15,000 to about 20,000. The pharmaceutically acceptable polymer may be Kollidan® VA 64 (copovidone, vinylpyrrolidone-vinyl acetate) having a CAS Number of 25086-89-9.

3. Excipients

In some embodiments, the core comprises an amorphous solid dispersion (ASD) excipient. In some aspects, the core further comprises a first core excipient, a second core excipient, a third core excipient, a fourth core excipient, and/or a fifth core excipient. In some embodiments, the amount of the each of the ASD and core excipients in the composition is from about 0.001% w/w to about 50% w/w, from about 0.0025% w/w to about 40% w/w, or from about 0.005% w/w to about 30% w/w. In some aspects, one or more of the excipients is a vitamin or vitamin derivative, salt, a polysaccharide such as cellulose or starch, a lubricant, a filler, a plasticizer, or an anti-foaming agent. In some aspects, a composition comprises a mixture of two or more excipients including two or more surfactants.

Exemplary ASD excipients include a vitamin or vitamin derivative. In some aspects, the vitamin derivative is a pegylated version of a vitamin, such as, for example, a vitamin E derivative. In some aspects, the vitamin derivative is D-α-tocopheryl polyethylene glycol succinate. In some embodiments, the amount of the ASD excipient in the composition is from about 0.5% w/w to about 10% w/w, from about 1% w/w to about 5% w/w, or from about 2% w/w to about 4% w/w, or any range derivable therein. In some embodiments, the amount of the ASD excipient in the composition is from about 0.1%, 0.25%, 0.5%, 1%, 1.5%, 2%, 2.5%, 3%, 3.5%, 4%, 4.5%, 5%, 5.5%, 6%, 6.5%, 7%, 7.5%, 8%, 9%, 10%, 11%, 12%, 12.5%, to about 15% w/w, or any range derivable therein.

In some aspects, the amount of the one, two, three, four, five, or more core excipients in the composition is from about 0.05% to about 30% w/w, from about 0.1% to about 25% w/w, from about 0.2% to about 20% w/w, or from about 5% to about 20% w/w, or any range derivable therein. The amount of the one, two, three, four, five, or more core excipient in the composition comprises from about 0.05%, 0.1%, 0.2%, 0.25%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 9%, 10%, 12%, 14%, 16%, 18%, 20%, 22%, 24%, 25%, 26%, 28%, to about 30% w/w, or any range derivable therein, of the total composition.

Exemplary first core excipients include polysaccharides, such as, for example, a cellulose. In some aspects, the cellulose has a particle size from about 1 μm to about 500 μm, from about 10 μm to about 300 μm, or from about 15 μm to about 200 μm. In some aspects, the cellulose has a particle size from about 1 μm, 5 μm, 10 μm, 15 μm, 20 μm, 30 μm, 40 μm, 50 μm, 75 μm, 100 μm, 125 μm, 150 μm, 175 μm, 200 μm, 350 μm, 400 μm, 450 μm, to about 500 μm , or any range derivable therein. In some aspects, the cellulose has a bulk density of from about 0.05 g/cm³ to about 1.0 g/cm³, from about 0.1 g/cm³ to about 0.75 g/cm³, or from about 0.2 g/cm³ to about 0.5 g/cm³. In some aspects, the cellulose has a bulk density of from about 0.01 g/cm³, 0.025 g/cm³, 0.05 g/cm³, 0.075 g/cm³, 0.1 g/cm³, 0.2 g/cm³, 0.25 g/cm³, 0.3 g/cm³, 0.4 g/cm³, 0.5 g/cm³, 0.6 g/cm³, 0.7 g/cm³, 0.75 g/cm³, 0.8 g/cm³, 0.9 g/cm³, to about 1.0 g/cm³, or any range derivable therein. In some embodiments, the amount of the first core excipient in the tablet is from about 5% w/w to about 30% w/w, from about 10% w/w to about 25% w/w, or from about 10% w/w to about 20% w/w, or any range derivable therein. In some embodiments, the amount of the first core excipient in the tablet is from about 1%, 1.5%, 2%, 2.5%, 3%, 3.5%, 4%, 4.5%, 5%, 5.5%, 6%, 6.5%, 7%, 7.5%, 8%, 9%, 10%, 12%, 14%, 16%, 18%, 20%, 22%, 24%, 25%, 26%, 28%, 30%, 32%, 34%, 36%, 38%, to about 40% w/w, or any range derivable therein.

Exemplary second core excipients include a modified cellulose derivative, or a modified starch derivative, or a vinylpyrrolidone copolymer. In some aspects, the modified cellulose derivative is a carboxymethyl cellulose. The modified cellulose derivative may be a cross linked carboxymethyl cellulose. The modified cellulose derivative may have a degree of substitution from about 0.4 to about 1.2, from about 0.5 to about 1.0, or from about 0.55 to about 0.9. In some aspects, the modified cellulose derivative may have a degree of substitution from about 0.1, 0.2, 0.3, 0.4, 0.5, 0.55, 0.6, 0.7, 0.8, 0.9, 1.0, 1.1, 1.2, 1.3, 1.4, to about 1.5, or any range derivable therein. In some aspects, the modified cellulose derivative is a monovalent metal salt, such as, for example, a sodium salt. In some aspects, the modified starch derivative comprises one or more carboxymethyl or a salt thereof. In some aspects, the salt is an alkali metal salt, such as, for example, a sodium salt. In some aspects, the modified starch derivative is sodium starch glycolate. In some aspects, the vinylpyrrolidone copolymer is a 1-vinyl-2-pyrrolidone and vinyl acetate copolymer, such as, for example, copovidone. In some embodiments, the amount of the second core excipient in the composition is from about 1% w/w to about 20% w/w, from about 2.5% w/w to about 15% w/w, or from about 5% w/w to about 10% w/w, or any range derivable therein. In some embodiments, the amount of the second core excipient in the composition is from about 0.1%, 0.5%, 1%, 1.5%, 2%, 2.5%, 3%, 3.5%, 4%, 4.5%, 5%, 5.5%, 6%, 6.5%, 7%, 7.5%, 8%, 9%, 10%, 12%, 14%, 16%, 18%, 20%, 22%, 24%, 25%, 26%, 28%, to about 30% w/w, or any range derivable therein.

Exemplary third core excipients include a salt, such as a sodium salt. In some aspects, the sodium salt is sodium chloride or sodium bicarbonate. In some embodiments, the amount of the third core excipient in the composition is from about 2.5% w/w to about 20% w/w, from about 5% w/w to about 15% w/w, or from about 8% w/w to about 12% w/w, or any range derivable therein. In some embodiments, the amount of the third core excipient in the composition is from about 1%, 1.5%, 2%, 2.5%, 3%, 3.5%, 4%, 4.5%, 5%, 5.5%, 6%, 6.5%, 7%, 7.5%, 8%, 9%, 10%, 12%, 14%, 16%, 18%, 20%, 22%, 24%, 25%, 26%, 28%, to about 30% w/w, or any range derivable therein.

Exemplary fourth core excipients include lubricants. In some aspects, the lubricant is a hydrophobic lubricant. In some aspects, the lubricant is a hydrophilic lubricant. In some aspects, the lubricant is a fatty acid, a salt of a fatty acid, sodium lauryl sulfate, magnesium stearate, magnesium silicate, calcium stearate, sodium lauryl sulphate, sodium stearyl fumarate, magnesium lauryl sulphate, stearic acid, calcium stearate, glyceryl behenate, behenoyl polyoxylglycerides, glyceryl dibehenate, lauric acid, glyceryl monostearate, glyceryl tristearate, myristic acid, palmitic acid, poloxamer, polyethylene glycol, polyethylene glycol 3350, polyoxyl 10 oleyl ether, polyoxyl 15 hydroxystearate, polysorbate 40, polyoxyl 20 cetostearyl ether, polyoxyl 40 stereate, potassium benzoate, sodium benzoate, sorbitan monolaurate, sorbitan monooleate, sodium stearate, sorbitan monopalmitate, sorbitan monostearate, zinc stearate, sorbitan sesquioleate, sorbitan trioleate, or talc, or any combination thereof.

In some aspects, the lubricant is a derivative or salt of stearic acid, such as, for example, magnesium stearate. In some embodiments, the amount of the fourth core excipient in the composition is from about 0.05% w/w to about 1% w/w, from about 0.1% w/w to about 0.5% w/w, or from about 0.2% w/w to about 0.3% w/w, or any range derivable therein. In some embodiments, the amount of the fourth core excipient in the composition is from about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, to about 3% w/w, or any range derivable therein.

Exemplary fifth core excipient include fillers, such as, for example, talc or silicone dioxide (e.g., colloidal silicone dioxide).

Each of the above excipient descriptions is meant to be exemplary in nature. Further examples of excipients are given below in a section entitled “Excipients,” which is specifically incorporated herein by reference.
B. Delayed Release Coating

In one embodiment, the core is encapsulated with a delayed release coating. The delayed release coating may essentially, substantially, or entirely encapsulate the core or the core in combination with the sealing coating in aspects where the composition has a sealing coating. In some aspects, the amount of the delayed release coating on the composition is from about 0.5% to about 15% w/w, from about 1% to about 10% w/w, or from about 2.5% to about 7.5% w/w. The amount of the one, two, three, or more delayed release excipient in the composition comprises from about 0.1%, 0.2%, 0.3%, 0.4%, 0.5%, 0.6%, 0.7%, 0.8%, 0.9%, 1.0%, 1.1%, 1.2%, 1.25%, 1.3%, 1.4%, 1.5%, 1.6%, 1.7%, 1.8%, 1.9%, 2.0%, 2.2%, 2.4%, 2.5%, 2.6%, 2.8%, 3.0%, 3.5%, 4.0%, 5%, 6%, 7%, 7.5%, 8%, 9%, 10%, 12%, 14%, 15%, 16%, 18%, to about 20% w/w, or any range derivable therein, of the total tablet.

In one embodiment, the delayed release coating comprises a cellulose polymer, such as, for example, an ethyl cellulose, hypromellose phthalate, or hypromellose acetate succinate. In one embodiment, the delayed release coating comprises an acrylate copolymer, such as, for example, a copolymer of methacrylate and an acrylic acid. In some aspects, the acrylate copolymer is a copolymer of methacrylate and ethyl acrylate comprising a ratio of methacrylate units and acrylic acid units of from about 10:1 to about 1:10, about 5:1 to about 1:5, about 2:1 to about 1:2, or about 1:1.

In some aspects, the delayed release coating further comprises a first delayed release coating excipient. The first delayed release coating excipient may be a plasticizer, such as, for example, citric acid or an ester of citric acid. One exemplary ester of citric acid is triethyl citrate. In some aspects, the amount of the first delayed release coating excipient in the composition is from about 0.05% to about 2.5% w/w, from about 0.1% to about 1.5% w/w, or from about 0.25% to about 1% w/w. The amount of the first delayed release coating excipient in the composition comprises from about 0.01%, 0.02%, 0.03%, 0.04%, 0.05%, 0.06% 0.07%, 0.08%, 0.09%, 0.1%, 0.2%, 0.3%, 0.4%, 0.5%, 0.6%, 0.7%, 0.8%, 0.9%, 1.0%, 1.2%, 1.4%, 1.6%, 1.8%, 2.0%, 2.2%, 2.4%, 2.5%, 2.6%, 2.8%, 3.0%, 3.5%, to about 4% w/w, or any range derivable therein, of the total composition.

In some aspects, the delayed release coating further comprises a second delayed release coating excipient. The second delayed release coating excipient may be an anti-foaming agent, such as, for example, siloxane. In some aspects, the anti-foaming agent further comprises silicon dioxide, such that the second delayed release coating excipient is, for example, a mixture of a siloxane and silicon dioxide. In some aspects, the anti-foaming agent is simethicone. In some aspects, the amount of the second delayed release coating excipient in the composition is from about 0.001% to about 0.1% w/w, from about 0.0025% to about 0.075% w/w, or from about 0.0025% to about 0.025% w/w. The amount of the second delayed release coating excipient in the composition comprises from about 0.001%, 0.002%, 0.0025%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, to about 0.5% w/w, or any range derivable therein, of the total composition.
C. Sealing Coating

In one embodiment, the core is encapsulated with a sealing coating. The sealing coating may essentially, substantially, or entirely encapsulate the core. The sealing coating, when present, is positioned between the core and the delaying release coating, such that the sealing coating encapsulates the core and then the sealing-coated core is encapsulated with the delayed release coating.

In one embodiment, the sealing coating comprises a polyvinyl alcohol or a cellulose polymer. In some aspects, the sealing coating further comprises a sealing coating excipient. The sealing coating excipient may be titanium oxide or a polyethylene glycol. The polyethylene glycol may comprise an average molecular weight from about 1,000 daltons to about 10,000 daltons, from about 2,000 daltons to about 5,000 daltons, from about 3,000 daltons to about 4,000 daltons. The polyethylene glycol may comprise an average molecular weight from about 1,000, 2,000, 3,000, 4,000, 5,000, 6,000, 7,000, 8,000, 9,000, to about 10,000 daltons, or any range derivable therein.

In some aspects, the amount of the sealing coating excipient in the composition is from about 0.1% to about 10% w/w, from about 0.5% to about 5% w/w, or from about 1% to about 3% w/w. The amount of the sealing coating excipient in the composition comprises from about 0.01%, 0.02%, 0.05%, 0.1%, 0.2%, 0.3%, 0.4%, 0.5%, 0.6%, 0.7%, 0.8%, 0.9%, 1.0%, 1.2%, 1.4%, 1.5%, 1.6%, 1.8%, 2.0%, 2.2%, 2.4%, 2.5%, 2.6%, 2.8%, 3.0%, 3.5%, 4.0%, 4.5%, 5%, 6%, 7%, 8%, 9%, 10%, 12%, 14%, to about 15% w/w, or any range derivable therein, of the total composition.

### II. Excipients

In some aspects, the present disclosure refers to one or more excipients formulated into compositions. An “excipient” refers to a pharmaceutically acceptable carrier that is a relatively inert substance used to facilitate administration or delivery of an active pharmaceutical ingredient (API) into a subject or used to facilitate processing of an API into drug formulations that can be used pharmaceutically for delivery to the site of action in a subject. Non-limiting examples of excipients include disintegrating agents, stabilizing agents, surfactants, surface modifiers, solubility enhancers, buffers, encapsulating agents, antioxidants, preservatives, nonionic wetting, fillers/diluents, or clarifying agents, viscosity increasing agents, and absorption-enhancing agents. In some aspects, one or more of the excipients is a vitamin or vitamin derivative, salt, a polysaccharide (e.g., cellulose or starch), a lubricant, a filler, a plasticizer, or an anti-foaming agent. In some aspects, the excipients used herein are water soluble and water insoluble excipients. These water-soluble or swellable excipients include a polysaccharides such as cellulose or starch. In some aspects, the cellulose or starch may comprise one or more modifications such as a carboxymethyl or ester thereof or one or more ether groups. Examples of starches and cellulose that may be used herein include a carboxymethyl cellulose, cross linked carboxymethyl cellulose, a monovalent metal salt of a cellulose derivative, sodium starch glycolate, crospovidone, croscarmellose sodium, low-substituted hydroxypropyl cellulose, sodium starch glycolate, chitosan hydrochloride, corn starch and pregelatinized starch, calcium alginate, calcium sodium alginate, docusate sodium, microcrystalline cellulose, hydroxypropyl starch, magnesium aluminum silicate, methylcellulose, sodium alginate, starch, calcium carboxymethylcellulose, calcium cellulose glycolate, carmellosum calcium, powdered cellulose, anhydrous lactose, lactose monohydrate, spray-dried lactose, mannitol, pregelatinized starch, maize starch, corn starch, sorbitol, sucrose, compressible sugar, sugar spheres, dextrates, dextrin, dextrose, calcium phosphate, calcium carbonate, maltose, maltodextrin, kaolin, calcium sulfate, cellaburate, calcium lactate, cellulose acetate, silicified microcrystalline cellulose, cellulose acetate, corn syrup, corn syrup solids, erythritol, ethylcellulose, ethyl acrylate and methyl methacrylate copolymer dispersion, fructose, isomalt, alpha-lactalbumin, lactitol, magnesium carbonate, magnesium oxide, methacrylic acid and ethyl acrylate copolymer, methacrylic acid and methyl methacrylate copolymer, polydextrose, sodium chloride, simethicone, pregelatinized modified starch, hydroxypropyl pea starch, potato starch, pregelatinized hydroxypropyl potato starch, wheat starch, pullulan, talc, amino methacrylate copolymer, trehalose, xylitol, In some aspects, the amount of each excipients in the composition is from about 5% to about 30% w/w, from about 10% to about 25% w/w, or from about 10% to about 20% w/w. The amount of this excipient in the composition comprises from about 5%, 5.5%, 6%, 6.5%, 7%, 7.5%, 8%, 9%, 10%, 12%, 14%, 16%, 18%, 20%, 22%, 24%, 25%, 26%, 28%, to about 30% w/w, or any range derivable therein, of the total composition.

### II. Manufacturing Methods

A. Hot Melt Extrusion

Thus, in one aspect, the present disclosure provides pharmaceutical compositions which may be prepared using a thermal or fusion-based high energy process. Such process may include hot melt extrusion, hot melt granulation, melt mixing, spray congealing, sintering/curing, injection molding, or a thermokinetic mixing process such as the KinetiSol method. Similar thermal processing methods are described in LaFountaine et al., 2016a, Keen et al., 2013, Vynckier et al., 2014, Lang et al., 2014, Repka et al., 2007, Crowley et al., 2007,

DiNunzio et al., 2010a, DiNunzio et al., 2010b, DiNunzio et al., 2010c, DiNunzio et al., 2010d, Hughey et al., 2010, Hughey et al., 2011, LaFountaine et al., 2016b, and Prasad et al., 2016, all of which are incorporated herein by reference. In some embodiments of these present disclosure, the pharmaceutical compositions may be prepared using a thermal process such as hot melt extrusion or hot melt granulation. In other embodiments, a fusion based process including thermokinetic mixing process such as those described at least in U.S. Pat. Nos. 8,486,423 and 9,339,440, the entire contents of which are herein incorporated by reference.

A non-limiting list of instruments which may be used to thermally process the pharmaceutical compositions described herein include hot melt extruders available from ThermoFisher®, such as a minilab compounder, or Leistritz®, such as a twin-screw extruder. Alternatively, a fusion-based high energy process instrument that does not require external heat input, including such as a thermokinetic mixer as described in U.S. Pat. Nos. 8,486,423 and 9,339,440 may be used to process the pharmaceutical composition.

In some aspects, the extruder may comprise heating the composition to a temperature from about 60° C. to about 300° C. In some embodiments, the temperature is from about 150° C. to about 250° C. The temperature that may be used is from about 60° C., 65° C., 70° C., 75° C., 80° C., 90° C., 92° C., 94° C., 96° C., 98° C., 100° C., 102° C., 104° C., 106° C., 108° C., 110° C., 112° C., 114° C., 116° C., 118° C., 120° C., 125° C., 130° C., 135° C., 140° C., 145° C., 150° C., 155° C., 160° C., 165° C., 170° C., 175° C., 180° C., 190° C., 200° C., 210° C., 220° C., 225° C., 230° C., 240° C., 250° C., 260° C., 270° C., 280° C., 290° C., to about 300° C. or any range derivable therein.

The extrudate produced following the extrusion process will generally comprise the active agent and the pharmaceutically acceptable polymer. The extrudate may be in the form of granules of a desired mesh size or diameter, rods that can be cut and shaped into tablets, and films of a suitable thickness that shaped forms can be punched into suitable size and shape for administration. This extrudate may be used in further processing steps to yield the final pharmaceutical product or composition. The extrudate of the pharmaceutical composition may be dried, formed, milled, sieved, or any combination of these processes to obtain a final composition which may be administered to a patient. Such processes are routine and known in the art and include formulating the specific product to obtain a final pharmaceutical or nutraceutical product. Additionally, the extrudate of the pharmaceutical composition obtained may be processed using a tablet press to obtain a final tablet. Additionally, it may be milled and combined with one or more additional excipients to form a capsule or pressed into a tablet. The resultant pharmaceutical composition may also be dissolved in a solvent to obtain a syrup, a suspension, an emulsion, or a solution.
B. Tablet Formation

As used herein, the niclosamide tablets may be formulated using a tablet press.

The tablets, in particular the core of the tablets, may be formed using either a single-punch or a rotary tablet press. Once the core is prepared using a tablet press, the core may be coated using a tablet coater. The tablet coaters that may be used include a standard coating pan, a perforated pan, or a fluidized bed or air suspension system.

### III. Definitions

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

By “delayed release” is meant that initial release of drug occurs after expiration of an approximate delay (or lag) period. For example, if release of drug from a composition is delayed two hours, then release of the drug begins at about two hours after administration of the composition, or dosage form, to a subject. In general, a delayed release is opposite of an immediate release, wherein release of drug begins after no more than a few minutes after administration.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis.

Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified polysaccharide, wherein at least one of the monomeric saccharide units is modified by substitution of atoms or molecular groups or bonds. In one embodiment, a derivative thereof is a salt thereof.

Salts are, for example, salts with suitable mineral acids, such as hydrohalic acids, sulfuric acid or phosphoric acid, for example hydrochlorides, hydrobromides, sulfates, hydrogen sulfates or phosphates, salts with suitable carboxylic acids, such as optionally hydroxylated lower alkanoic acids, for example acetic acid, glycolic acid, propionic acid, lactic acid or pivalic acid, optionally hydroxylated and/or oxo-substituted lower alkanedicarboxylic acids, for example oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example methanesulfonates, benzenesulfonates, p-toluenesulfonates or N-cyclohexylsulfamates (cyclamates).

The term “dissolution” as used herein refers to a process by which a solid substance, here the active ingredients, is dispersed in molecular form in a medium. The dissolution rate of the active ingredients of the pharmaceutical dose of the invention is defined by the amount of drug substance that goes in solution per unit time under standardized conditions of liquid/solid interface, temperature and solvent composition.

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active agent in the composition is measured by powder x-ray diffraction.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate that a value includes the inherent variation of error for the device, the method being employed to determine the value, or the variation that exists among the study subjects or experimental studies. Unless another definition is applicable, the term “about” refers to ±5% of the indicated value.

As used herein, the term “substantially free of” or “substantially free” in terms of a specified component, is used herein to mean that none of the specified component has been purposefully formulated into a composition and/or is present only as a contaminant or in trace amounts. The total amount of all containments, by-products, and other material is present in that composition in an amount less than 2%. The term “essentially free of” or “essentially free” is used to represent that the composition contains less than 1% of the specific component. The term “entirely free of” or “entirely free” contains less than 0.1% of the specific component.

As used herein, the term “substantially” when used in the context of encapsulation means largely but not wholly, or that less than about 5% of the element to be encapsulated is not covered by the element encapsulating it. The term “essentially” when used in the context of encapsulation means that less than about 1% of the element to be encapsulated is not covered by the element encapsulating it. The term “entirely” when used in the context of encapsulation means that less than 0.1% of the element to be encapsulated is not covered by the element encapsulating it.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. Examples

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Oral Formulations of Niclosamide as Amorphous Solid Dispersion

### A. Preparation of Oral Formulation

The formulations were processed using a HAAKE Minilab II microextruder with a screw speed of 150 rpm at 180° C. The cooled extrudates were milled into granules and particles retained on a 45 μm sieve but that passed through a 150 μm sieve were used for further testing. Dissolution tests were conducted on polymer-niclosamide extrudates at different drug loadings in a Hanson SR8-Plus apparatus (Hanson Research Co., USA) using the 200 mL vessels and their paddles. FaSSIF medium (Biorelevant.com Ltd., UK) was prepared according to manufacturer specifications. Formulations containing 80 mg of niclosamide were added into 150 mL of FaSSIF medium, the apparatus was set at 37.0±0.5° C. and 100 rpm. The sampling times were 5, 10, 15, 30, 60, and 120 minutes. When recollecting the samples, these were passed through 0.2 μm filters. Then, 0.5 mL of the samples were mixed with 1 mL of acetone and 0.5 mL of acetonitrile for HPLC analysis.

### B. HPLC Analysis

The samples were measured at 331 nm using a Dionex® HPLC system (Thermo Fisher® Scientific Inc. , USA) with a ZORBAX® SB-C18 column (4.6×250 mm, 5 μm) (Agilent®, USA) at 1 mL/min flow rate. Two mobile phases were used, the mobile phase A was formic acid water solution at 0.3%, and the mobile phase B was acetonitrile, they were mixed in a 40:60 ratio.

### C. Dissolution Analysis for Oral Formulations

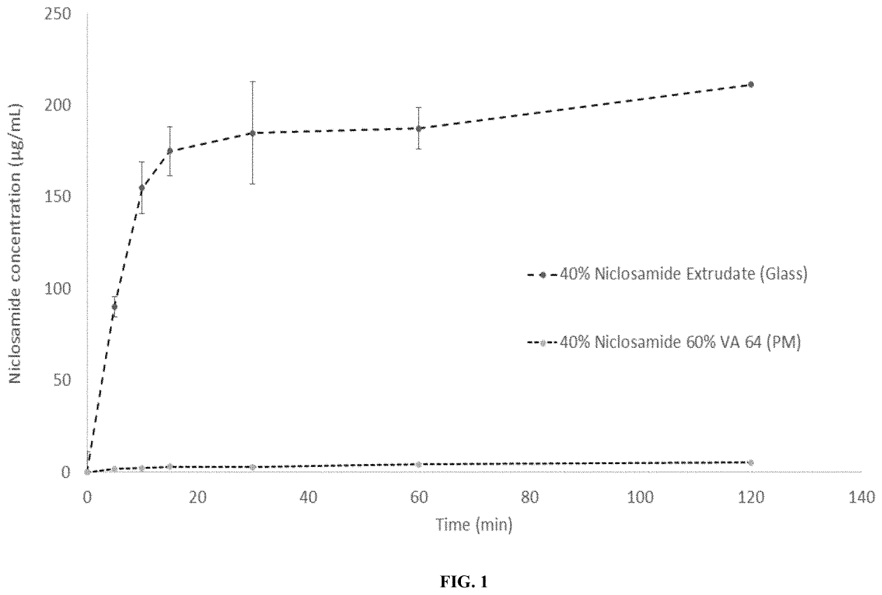

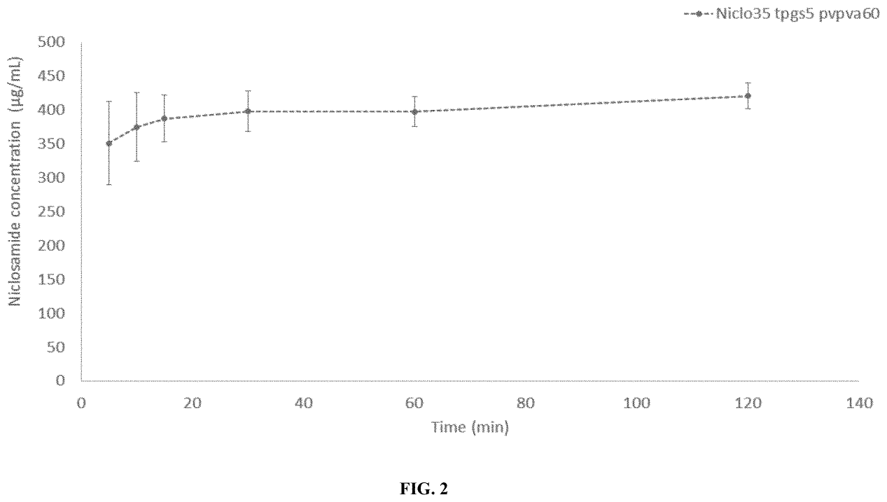

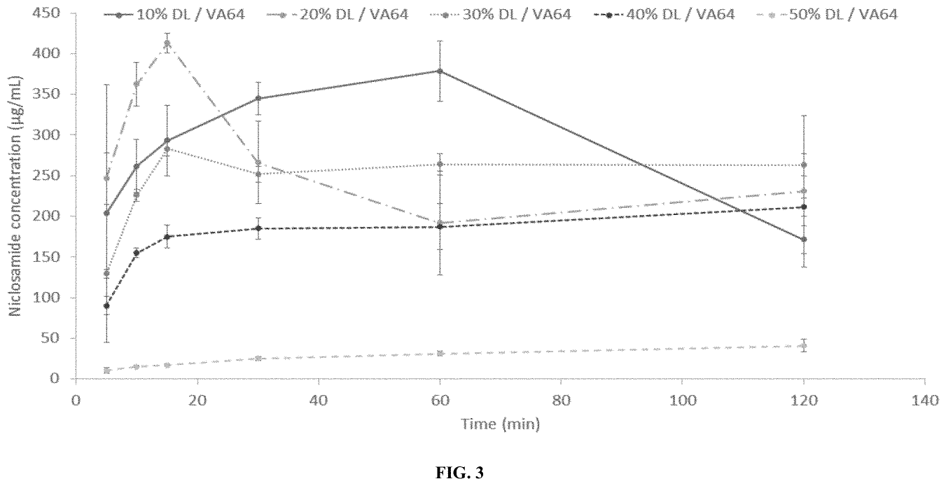

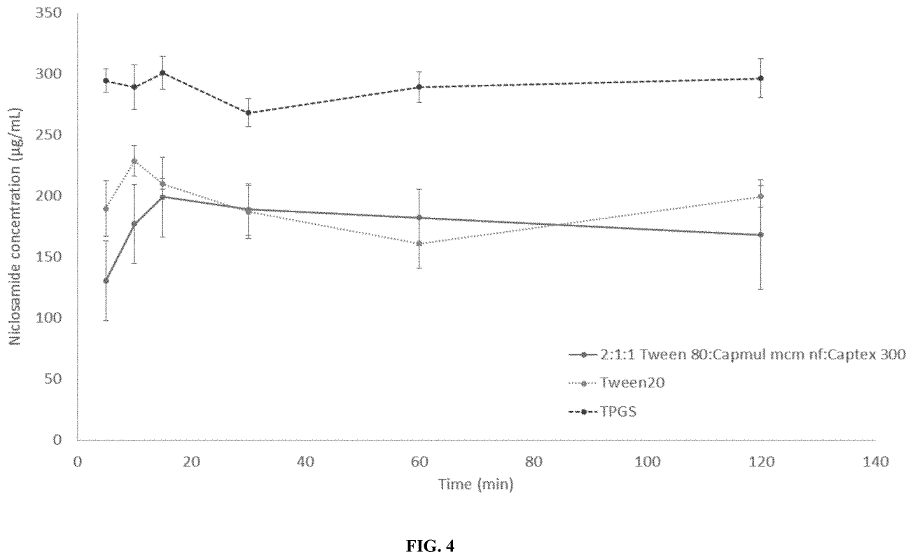

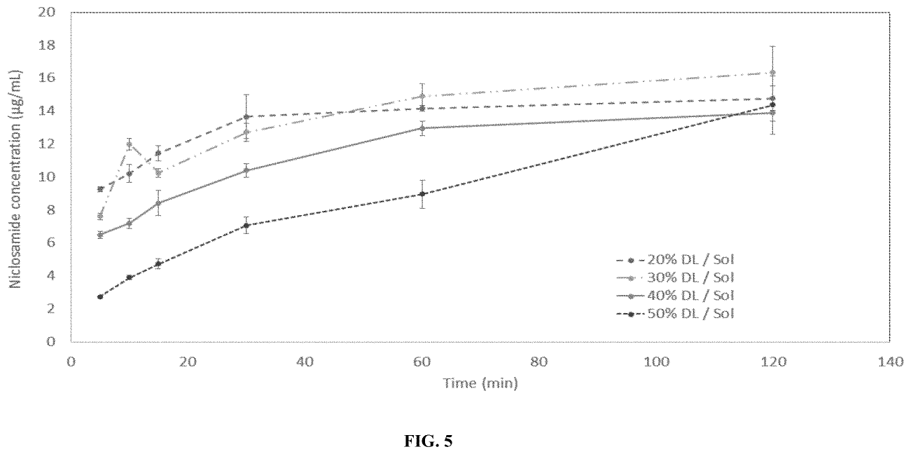

A sample of Kollidon VA64® with niclosamide was physically mixed and another sample was process through a hot melt extruder as described in above. These two samples were subjected to HPLC analysis to determine the dissolution of the drug in the composition. The HPLC analysis showing significant dissolution of niclosamide into the composition are shown in FIG. 1. Then, the amount of drug in the composition was varied between 10% drug load and 50% drug load which all showed high dissolution of niclosamide in the extrudate (FIG. 3). Several surfactant compositions were tested to determine their ability to maintain the niclosamide concentration in the dissolution assay. FIG. 4 shows that all three composition showed the ability to retain the niclosamide but the composition with TPGS showed the highest overall dissolution. Finally, decreasing the drug load to 35% while maintaining TPGS at 5% of showed high dissolution which persisted over 2 hours. See FIG. 2. Two other polymer excipients were tested (Kollidon 30® and SolvPlus®) which showed dissolution of niclosamide but at a lower extent than Kollidon VA64® (FIGS. 5 & 6).

### D. Pharmacokinetic Evaluation of Milled Niclosamide Extrudate

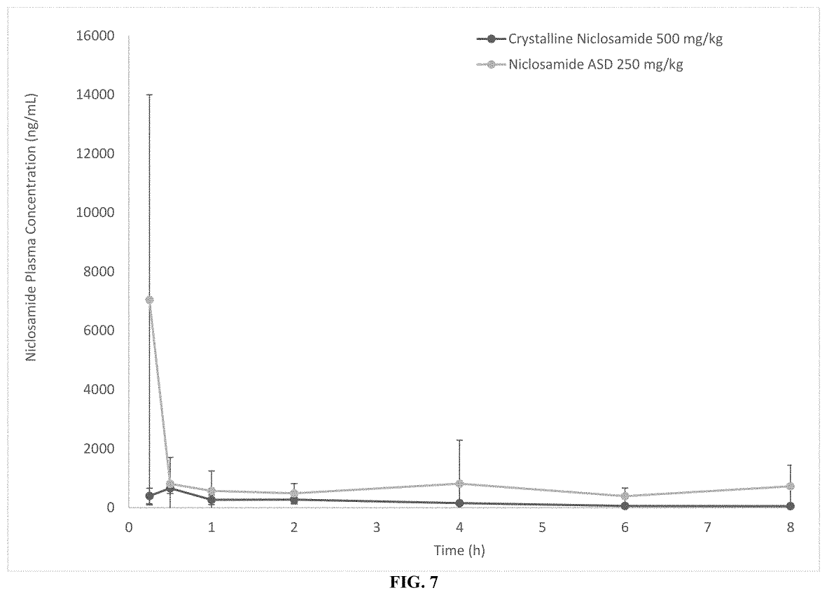

CD-1 mice were dosed with either crystalline niclosamide at a dose of 500 mg/kg suspended in 0.5% Methocel A4M in a volume corresponding to 10 ml/kg or with milled niclosamide extrudate at a dose of 250 mg/kg suspended in pH 6.5 FaSSIF media (Biorelevant.com Ltd) in a volume corresponding to 10 mL/kg. The pH 6.5 FaSSIF media was prepared as instructed with the exception of the buffer salts, sodium hydroxide and sodium phosphate monobasic anhydrous, being present at two-fold their instructed concentration. Plasma samples from mice were taken at 0.25, 0.5, 1, 2, 4 6, and 8 h after administration and analyzed by LC/MS/MS for niclosamide concentration. See the data shown in FIG. 7 and Table 1.

TABLE 1
Pharmacokinetic Data for ASD formulation of Nicolsamide

| | Crystalline Nic (500 | Nic ASD |
|---|---|---|
| PK Parameter | mg/kg) | (250 mg/kg) |
| Cmax (ng/mL) | 661.7 | 7048.0 |
| Tmax (h) | 0.5 | 0.25 |
| AUC0-last | 1454.6 | 6375.5 |
| (ng*h/mL) | | |

### E. Particle Size Analysis During Dissolution

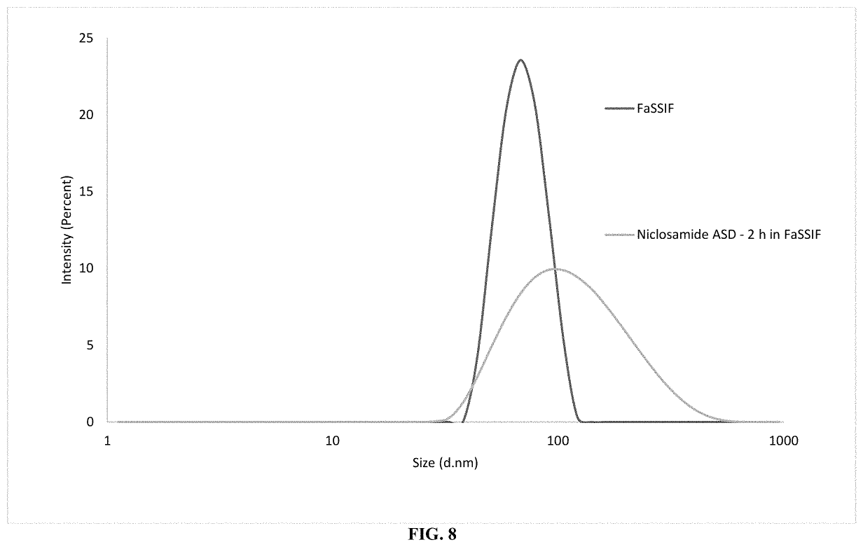

Samples taken from the dissolution vessel were centrifuged at 13,000 rpm (14,300 rcf) for 10 min. Then, the supernatant was measured using a Zetasizer Nano ZS (Malvern Instruments Ltd., Worcestershire, UK). The dispersant was water, and the samples were equilibrated at 37° C. before being measured using the 173° backscatter with automatic measurement duration in triplicate (FIG. 8).

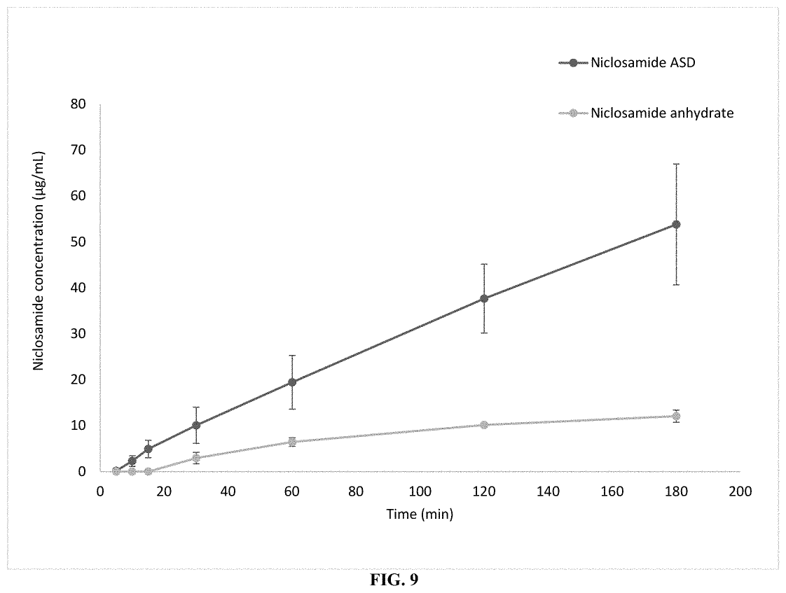

Side-by-side diffusion cells (PermeGear, Hellertown, Pa., USA) were employed to evaluate the diffusion of the niclosamide ASD through a 0.03 μm polyethersulfone membrane (Sterlitech Corp., Kent, Wash., USA). The donor and receiver cells were filled with 34 mL of FaSSIF and decanol, respectively. 52.1 mg of the niclosamide ASD and 18.2 mg of niclosamide anhydrate was added to the donor cell at 37° C. and 850 rpm. The samples were collected from the receiver cell at 5, 10, 15, 30, 60, 120, and 180 min. Samples were measured using the same HPLC method described above. See FIG. 9.

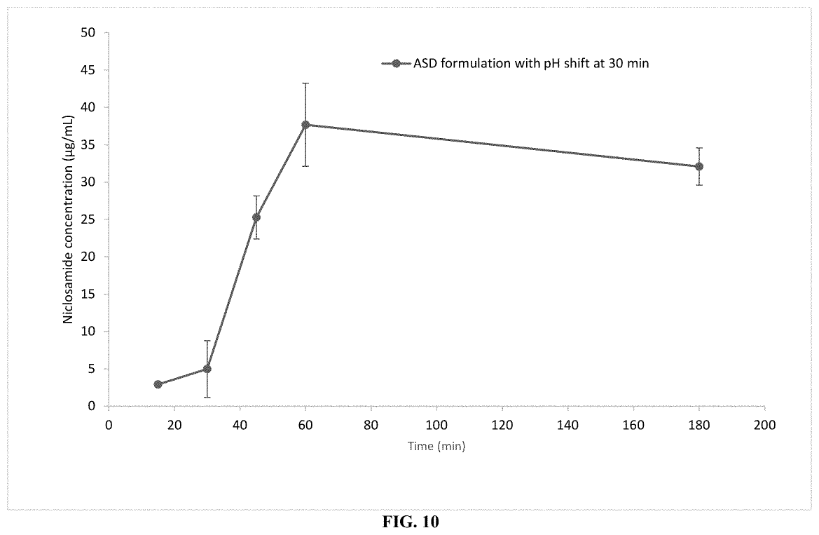

The pH-shift dissolution tests were performed using the same equipment in two stages. First, 230 mg of niclosamide ASD was poured in 30 mL of HCl 0.01 M for 30 min. Thereafter, 150 mL of FaSSIF was added into the vessel, completing a volume of 180 mL, and the samples were taken at the same time points of the previously described dissolution test. When required, to separate the particles and the unbound drug from the samples, an Airfuge™ Air-Driven Ultracentrifuge (Beckman Coulter, Palo Alto, Calif., USA) was used at 30 psi for 30 min. Then, the supernatant was measured using HPLC. See FIG. 10.

### F. Animal Studies

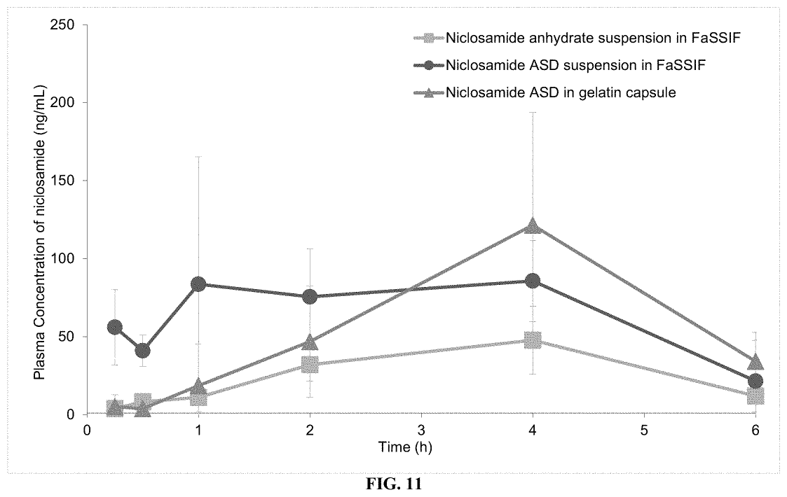

The oral pharmacokinetic analysis was conducted at Pharmaron (Ningbo, China). The study protocol was approved and conducted in accordance with the Institutional Animal Care and Use Committee (IACUC) guidelines at Pharmaron. (IACUC; Protocol Number AUP-PK-R-06012019). In this study, niclosamide anhydrate and niclosamide ASD were administered to five rats per group (weight =205.8±2.9 g each) at a niclosamide dose of 10 mg/kg by oral gavage. The groups received a FaSSIF suspension of niclosamide anhydrate at 1.5 mg/mL, a FaSSIF suspension of niclosamide ASD at 1.5 mg/mL, and size 9 mini capsules (Braintree Scientific, Braintree, Mass., USA) containing niclosamide ASD, respectively (three groups in total). In this last group, the capsule size 9 contained 60% niclosamide ASD, 15% EXPLOTAB©, and 25% sodium bicarbonate. The powders were blended by mortar and pestle and loaded into the capsules using the capsule filling funnel for size 9 (Torpac, Fairfield, N.J., USA). The samples were measured using an AB Sciex Triple Quad 5500 LC/MS/MS with an Agilent Eclipse® XDB-C18 column (2.1×150 mm, 5 μm) (Agilent, Palo Alto, Calif., USA) at a flow rate of 0.6 mL/min. Two mobile phases were used. The mobile phase A was a 0.1% formic acid aqueous solution, and the mobile phase B was a mixture of 5% water and 95% acetonitrile (0.1% formic acid). They were mixed as shown in Table 2. Then, 50 μL of plasma with 5 μL of methanol were added to 200 μL of methanol containing an internal standard mixture for protein precipitation. The samples were vortexed for 30 s and underwent centrifugation for 15 min at 4000 rpm and ° C. Thereafter, the supernatant was diluted three times with water, and 2 μL were injected into the HPLC. The results of the pharmacokinetic profile studies are shown in FIG. 11 and Table 3. Furthermore, the resultant particles were subjected to dissolution and the resultant supernatants after centrifugation were analyzed. From this data, it can be noted that dissolution in FaSSIF helps in the generation of smaller nanoparticles. See Table 4.

TABLE 2
Mobile phase gradient that was used for analyzing plasma sample.

| Time | A | B |
|---|---|---|
| (min) | (%) | (%) |
| 0.20 | 85.0 | 15.0 |
| 2.00 | 50.0 | 50.0 |
| 2.50 | 50.0 | 50.0 |
| 4.00 | 0.00 | 100 |
| 4.50 | 0.00 | 100 |
| 4.51 | 85.0 | 15.0 |
| 5.00 | 85.0 | 15.0 |

TABLE 3
Pharmacokinetic parameter profiles (in rats) of niclosamide anhydrate
suspended in FaSSIF, niclosamide ASD suspended
in FaSSIF, and niclosamide ASD in capsules (n = 5).

| | Niclosamide | Niclosamide | |
|---|---|---|---|
| | anhydrate | ASD | Niclosamide |
| | suspension in | suspension | ASD in |
| PK parameters | FaSSIF | in FaSSIF | capsules |
| T_{1/2 (h)} | 1.00 (0.30) | 1.59 (1.34) | 0.84 (0.01) |
| T_{max (h)} | 3.60 (0.89) | 2.40 (1.52) | 4.40 (0.89) |
| C_{max (ng/mL)} | 48.3 (20.6) | 123 (56) | 122 (71) |
| AUC_{last(h*ng/mL)} | 168 (64) | 398 (115) | 338 (193) |
| AUC_{Inf(h*ng/mL)} | 188 (84) | 495 (239) | 463 (224) |
| AUC_%Extrap_obs (%) | 8.4 (7.0) | 12.2 (21.4) | 7.98 (0.48) |
| MRT_{Inf}_obs (h) | 3.56 (0.70) | 3.71 (2.20) | 4.04 (0.12) |
| AUCₗₐₛₜ/D (h*mg/mL) | 16.8 (6.4) | 39.8 (11.5) | 33.8 (19.3) |

TABLE 4
Mean particle size, PDI, and zeta potential of supernatants after
centrifugation at 13,000 rpm × 10 min. The samples were
taken from the dissolution apparatus at different time points.

| | Sampling | Mean | | Zeta |
|---|---|---|---|---|
| | time | particle | | potential |
| Sample | (h) | size (d · nm) | PDI | (mV) |
| FaSSIF media | 1 | 66.5 ± 0.9 | 0.037 ± | −14.8 ± 2.3 |
| | | | 0.048 | |
| Extrudate niclosamide | 1 | 228.1 ± 4.2 | 0.157 ± | −12.1 ± 0.1 |
| 35%-TPGS 5%-PVP | | | 0.011 | |
| 60% in Buffer 6.5 | | | | |
| Extrudate niclosamide | 1 | 99.3 ± 1.4 | 0.224 ± | −13.6 ± 1.0 |
| 35%-TPGS 5%-PVP | | | 0.004 | |
| 60% in FaSSIF | | | | |

### Example —Delayed Release Tablet Form of Niclosamide

### A. Preparation of 100 mg Niclosamide Delayed-Release Tablets

The tablet compositions are shown below.

TABLE 5
Composition per tablet

| Ingredient | % (w/w) | Mg/tablet |
|---|---|---|
| Niclosamide | 23.3 | 100 |
| Copovidone (Kollidon VA64) | 40.0 | 172 |
| Vitamin E TPGS | 3.33 | 14.3 |
| Microcrystalline Cellulose | 13.1 | 56.1 |
| PH200 | | |
| Croscarmellose sodium | 8.90 | 38.2 |
| Sodium Chloride | 11.1 | 47.8 |
| Magnesium Stearate | 0.28 | 1.19 |

TABLE 6
Step #1 Preparation of Niclosamide Milled Extrudate
to form Niclosamide ASD granules

| Component | Weight (g) |
|---|---|
| Niclosamide | 175 |
| Copovidone | 300 |
| Vitamin E TPGS | 25 |

Niclosamide and copovidone (Kollidon VA64) were weighed and placed in a high shear granulator. The granulator was pulsed several times until a uniform light yellow powder was present. Vitamin E TPGS was weighed into a 100 mL beaker and melted in a water bath at 60° C. With the high shear granulator mixing, Vitamin E TPGS was slowly poured into the mixer over a 3 minute period followed by an additional running of the mixer for 90 seconds. Then, the mixture was removed from the high shear granulator and passed through a Fitzmill L1A operating at 5000 rpm equipped with a 0.06″ round hole screen.

The prepared granulated mixture was then hot melt extruded using a 12 mm extruder (Leistritz). The extrusion parameters were as follows: Product feed rate of 3 g/min. with a screw speed of 50 rpm and operating temperatures depicted in the tablet below:

TABLE 7
Extrusion Parameters

| Zone | Feeder | 1 | 2 | 3 | 4 | 5 | 6 | 7 | Die |
|---|---|---|---|---|---|---|---|---|---|
| Temperature ° C. | 60 | 90 | 140 | 160 | 160 | 160 | 150 | 130 | 115 |

The extrudate was milled using a Fitzmill (model 1A) operated at 7551 rpm and using a round hole 0.02″ milling screen.

TABLE 8
Step #2 Preparation of Niclosamide Tablet Cores

| Component | Weight (g) |
|---|---|
| Niclosamide Milled Extrudate | 244.7 |
| Microcrystalline Cellulose (Ceolus PH-200) | 47.9 |
| Croscarmellose Sodium | 40.8 |
| Sodium Chloride | 32.6 |
| Magnesium Stearate | 1.02 |

Niclosamide milled extrudate, microcrystalline cellulose, croscarmellose sodium, and sodium chloride were weighed and blended in a mortar and pestle. Magnesium stearate was added and blended with the other ingredients. The blend was filled into a hopper placed on a Stoke F4 Press. Tablet cores were produced with a target final tablet weight of 428 mg.

TABLE 9
Step # 4 Niclosamide Delayed-Release Tablets

| | i | ii | iii | iv |
|---|---|---|---|---|
| Opadry | 3% wg (H₂O) | 3% wg | 3% wg | 3% wg |
| II Clear | | (aqueous) | (Ethanol/H₂0) | (Ethanol/H₂0) |
| Acryl | 5,7.5,10% wg | — | 5,7.5,10% wg | — |
| EZE 93 A | (H₂O) | | (H₂O) | |
| Opadry | — | 5,7.5,10% wg | — | 5,7.5,10% wg |
| Enteric | | (Ethanol/H₂0) | | (Ethanol/H₂0) |

wg = weight gain

Tablet cores were coated in with either a completely water based coating (i), or a mixture of water and organic solvent based coating (ii-iv) at weight gains (% wg) ranging from 5 to 10%.

### B. Preparation of 37.5 mg Niclosamide Delayed-Release Tablets

The quantitative composition for niclosamide delayed-release tablets is provided below:

TABLE 10
37.5 mg Tablet Composition

| Component | % weight/weight | Mg/Tablet |
|---|---|---|
| Niclosamide | 20.45 | 37.5 |
| Copovidone | 35.06 | 64.3 |
| Vitamin E TPGS | 2.92 | 5.4 |
| Microcrystalline Cellulose | 20.02 | 36.7 |
| (Avicel PH-102) | | |
| Croscarmellose Sodium | 8.20 | 15.0 |
| Sodium Chloride | 10.22 | 18.7 |
| Magnesium stearate | 0.26 | 0.5 |
| Total: | 97.1 | 178.1 |
| Opadry II Clear | 2.9 | 5.3 |
| Total: | 100.0 | 183.3 |
| Acryl EZE 93A | 7.50 | 13.73 |
| Triethyl Citrate | 0.90 | 1.65 |
| Simethicone | 0.01 | 0.02 |
| Total: | 108.4 | 198.7 |

The quantitative batch composition and preparation

TABLE 11
Step #1 Preparation of Niclosamide Milled Extrudate

| Component | Weight (g) |
|---|---|
| Niclosamide | 1575.0 |
| Copovidone | 2700.0 |
| Vitamin E TPGS | 225.0 |

Niclosamide is passed through a conical mill. Copovidone is sieved through a 20-mesh sieve followed by adding to a high shear mixer/granulator with the milled niclosamide.

The dry components are mixed in the mixer. Vitamin TPGS is melted and maintained at 60±5° C. With the mixer running, the melted Vitamin E TPGS is slowly added to the other components. The final component mixture is milled with a Fitzmill Comminutor equipped with a 0.065″ round screen and blades oriented for impaction. The milled blend of the 3 components is fed into a Leistritz ZSE 18HP Extruder at a rate of 0.60 kg/hr. The extruder barrel temperature is as follows running with a screw speed of 200 RPM:

TABLE 12
Extrusion Parameters

| | Zone | | | | | | | | | |
|---|---|---|---|---|---|---|---|---|---|---|
| | | | | | | | | | 8 | 9 |
| | Feed Zone | 1 | 2 | 3 | 4 | 5 | 6 | 7 | Melt Plate | Die Heater |
| Temperature Set Point ±5 (° C.) | Cooling | 60 | 90 | 140 | 160 | 160 | 160 | 150 | 150 | 150 |

The extrudate rods are milled with a Fitzmill comminutor equipped with a 0.020″ round screen operated at approximately 7500 rpm with blades oriented for impaction.

TABLE 13
Step # 2 Preparation of Niclosamide Tablet Cores

| Component | Weight (g) |
|---|---|
| Niclosamide Milled Extrudate | 1110.2 |
| Microcrystalline Cellulose (Avicel PH-102) | 380.4 |
| Croscarmellose Sodium | 155.8 |
| Sodium Chloride | 194.2 |
| Magnesium Stearate | 4.940 |

Microcrystalline cellulose, croscarmellose sodium, and sodium chloride were sieved through 20 Mesh screen. Microcrystalline cellulose, followed by Niclosamide Milled Extrudate, followed by Croscarmellose Sodium, followed by Sodium Chloride were charged into a Bohle LM 40 Blender. The components were blended for 10 minutes at 25 rpm. Magnesium stearate was sieved through a 20 mesh and charged to the blender. The blender was operated for 3 minutes at 25 rpm. The final blended components were compressed into tablet cores using a rotary tablet press.

TABLE 14
Step #3 Niclosamide Clear Coated Tablets

| Component | Weight (g) |
|---|---|
| Niclosamide Tablet Cores | 977.5 |
| Opadry II Clear | 82.7 |
| Purified Water, USP | 1098.7 |

Niclosamide Tablet Cores were added to a Vector LDCS-Hi Coater. The Opadry II Clear coating suspension was coated to a target weight gain of 3%.

TABLE 15
Step #4 Delayed-Release Niclosamide Tablets

| Component | Weight (g) |
|---|---|
| Niclosamide Clear Coated Tablets | 981.7 |
| Acryl EZE 93 A | 285.0 |
| Triethyl Citrate | 34.2 |
| Simethicone | 0.380 |
| Purified Water | 1278.3 |

Simethicone was added to the purified water and mixed for 5 minutes followed by the addition of Triethyl Citrate. After an additional 5 minutes of mixing the Acryl EZE 93A was slowly added under mixing and mixed for a minimum of 45 minutes. After mixing is complete, the suspension is passed through a 60 mesh screen prior to coating. Niclosamide Clear Coated Tablets were added to a Vector LDCS-Hi Coater. The Acryl EZE 93A suspension was coated onto the Niclosamide Clear Coated Tablets to a target weight gain of 7.5%.

### C. Measuring Release Profile for 100 mg Niclosamide Delayed-release Tablets

The amount of niclosamide released from niclosamide Delayed-release tablets was measured using a USP type II apparatus with low volume vessels (150 mL) and paddles at a speed of 100 rpm in two stages. The first stage of dissolution took place in 120 mL 0.1 N HCl at 37.0±0.5° C. with one tablet added to the dissolution vessel followed initiating the paddles to rotate at 100 rpm. After 2 hours a 2.0 mL sample was taken from the 0.1 N HCL and filtered through a 0.2 μm Nylon syringe filter for analysis by HPLC. The 0.1 N HCL media was removed and 150 mL of pH 6.5 FaSSIF (Biorelevant.com Ltd) was added to the vessel. Samples were taken after the change in media, filtered through a 0.2 μm Nylon filter, and analyzed by HPLC for niclosamide concentration.

### D. Dissolution Profile of 100 mg Delayed-Release Niclosamide Tablets

The dissolution profile for 100 mg niclosamide delayed-release tablets prepared in Example 2A using the method for measurement described in Example 2C is as follows (freshly made tablets, n=2):

| | | i (μg/mL) | | | iv (μg/mL) | | |
|---|---|---|---|---|---|---|---|
| Time | Media | 5% wg | 7.5% wg | 10% wg | 5% wg | 7.5% wg | 10% wg |
| 0 | 0.1N HCL | — | — | — | — | — | — |
| 120 | 0.1N HCL | 0.07 | 0.00 | 0.00 | 0.00 | 0.00 | 0.00 |
| 125 | FaSSIF | 0.02 | 0.03 | 0.04 | 0.00 | 0.00 | 0.00 |
| 130 | FaSSIF | 0.07 | 0.07 | 0.06 | 0.20 | 0.14 | 0.16 |
| 135 | FaSSIF | 40.14 | 44.27 | 0.68 | 0.27 | 0.26 | 0.23 |
| 150 | FaSSIF | 377.74 | 276.11 | 20.48 | 0.43 | 0.24 | 0.20 |
| 165 | FaSSIF | 465.15 | 394.69 | 279.64 | 175.57 | 35.57 | 6.18 |
| 180 | FaSSIF | 490.58 | 437.55 | 418.53 | 343.54 | 311.52 | 82.55 |

### E. Measuring Release Profile for 37.5 mg Niclosamide Delayed-release Tablets

The amount of niclosamide released from Niclosamide Delayed-release tablets was measured using a USP type II apparatus with low volume vessels (150 mL) and paddles at a speed of 100 rpm in two stages. The first stage of dissolution took place in 120 mL 0.1 N HCl at 37.0±0.5° C. with two tablets added to the dissolution vessel followed by initiating the paddles to rotate at 100 rpm. After 2 hours a 2.0 mL sample was taken from the 0.1 N HCL and filtered through a 0.2 μm Nylon syringe filter for analysis by HPLC. The 0.1 N HCL media was removed and 112 mL of pH 6.5 FaSSIF (Biorelevant.com Ltd) was added to the vessel. Samples were taken after the change in media, filtered through a 0.2 μm Nylon filter and analyzed by HPLC for niclosamide concentration.

### F. Dissolution Profile of 37.5 mg Delayed-Release Niclosamide Tablets

The dissolution profile of 37.5 niclosamide delayed-release tablets using the method for measurement described in Example 2E is as follows:

| | | Niclosamide Concentration |
|---|---|---|
| Time | Media | (μg/mL) |
| 0 | 0.1N HCL | — |
| 120 | 0.1N HCL | 0.00 |
| 125 | FaSSIF | 0.00 |
| 130 | FaSSIF | 23.65 |
| 135 | FaSSIF | 112.03 |
| 150 | FaSSIF | 235.01 |
| 165 | FaSSIF | 261.61 |
| 180 | FaSSIF | 280.32 |
| 200 | FaSSIF | 274.49 |

G. Composition of Enteric Capsules and Dissolution Tests using FaSSIF as a Biorelevant Medium

400 mg of 25% Sodium bicarbonate, 5% Explotab®, and 70% ASD are blended using a mortar and pestle and loaded into size 0. Then, the dissolution test is performed using the same equipment in two stages. The capsules are placed (with sinkers) in 30 mL of HCl 0.01M for 30 min. Thereafter, the media is changed for 150 mL of FaSSIF for 120 minutes

H. Composition of enteric-coated capsules size 9 and dissolution tests using FaSSIF as a biorelevant medium

25% of Sodium bicarbonate, 15% of Explotab®, and 60% of ASD are mortar and pestle until homogeneity. Then, the capsules Size 9 are loaded with 15 mg of the mixture and coat with an ethanolic solution of Eudragit L 100 12% w/w and Triethyl citrate 5% w/w according to the Size 9 & Size M Capsule Holder Instructions (Torpac, USA). Then, the capsules are placed (with sinkers) in 30 mL of HCl 0.01M for 30 min. Thereafter, the media is changed for 50 mL of FaSSIF for 120 minutes.

I. Composition of Enteric Capsules and Dissolution Tests using FaSSIF as a Biorelevant Medium.

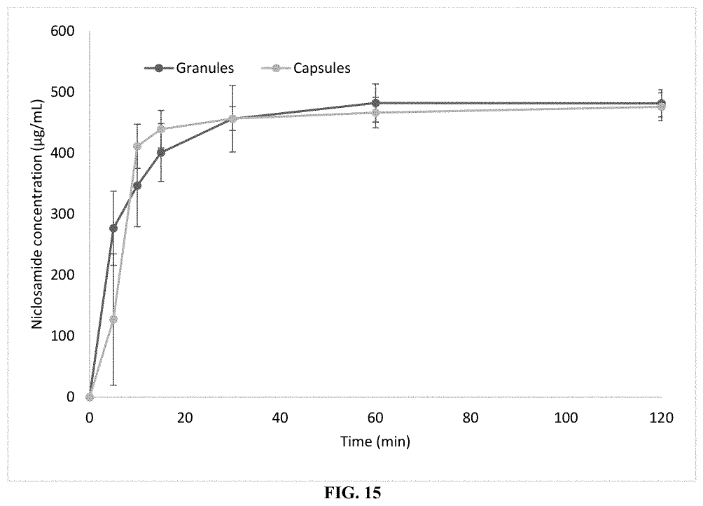

Niclosamide ASD granules were prepared as described in Example 2A. Encapsulated niclosamide ASD granules were prepared by admixing 400 mg of 25% Sodium bicarbonate, 5% Explotab®, and 70% ASD and filled into Capsuline capsules size 0 (HPMC and HPMCP). Then, dissolution testing was performed as described in the USP using a type II apparatus with low volume vessels (150 mL) and at 100 rpm in 150 mL of FaSSIF for 120 minutes at 37° C. (FIG. 15). When testing under these specific conditions of Example I, dissolution media did not cause undesired crystallization of the niclosamide in the composition.

J. pH-shift dissolution profile of enteric capsules containing Niclosamide ASD granules

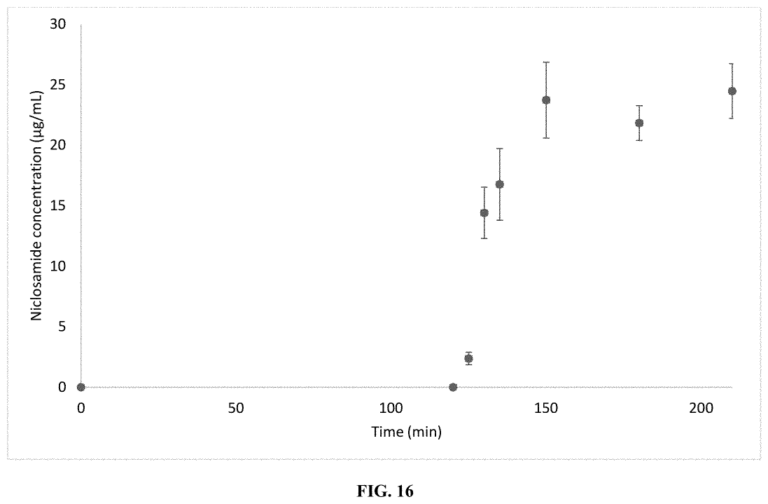

The amount of niclosamide released from enteric capsules (prepared from example 9) was measured using a USP type II apparatus with low volume vessels (150 mL) and paddles at a speed of 100 rpm in two stages. The first stage of dissolution took place in 120 mL 0.1 N HCl at 37.0±0.5° C., the enteric capsule (with sinker) was added to the dissolution vessel followed by initiating the paddles to rotate at 100 rpm. After 2 hours a 2.0 mL sample was taken from the 0.1 N HCL and filtered through a 0.2 μm Nylon syringe filter for analysis by HPLC. The 0.1 N HCL media was removed and 150 mL of pH 6.5 FaSSIF (Biorelevant.com Ltd) was added to the vessel. Samples were taken after the change in media, filtered through a 0.2 μm Nylon filter and analyzed by HPLC for niclosamide concentration (FIG. 16). When testing under these specific conditions of Example J, the enteric capsules did not protect the niclosamide ASD composition and allowed leakage of the acidic media inside the enteric capsule during the acid phase, which caused crystallization of niclosamide and subsequent failure.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### References

The following references, to the extent that they provide exemplary procedural or other details supplementary to those set forth herein, are specifically incorporated herein by reference.

U.S. Pat. No. 8,968,786
U.S. Patent App. No. 2010/0221343

### Ahmed et al., Viruses, 12:254, 2020.

Barbosa et al., Eur. J. Pharm. Biopharm., 141:58-69, 2019.
Beinborn et al., European journal of pharmaceutics and biopharmaceutics, 81(3):600-8, 2012a.

### Beinborn et al., Int J Pharmaceut., 429(1-2):46-57, 2012b.

### Bray et al., CA: A Cancer Journal For Clinicians, 68(6):394-424, 2018.

Carvalho et al., European journal of pharmaceutics and biopharmaceutics, 88(1):136-47, 2014.
Chen et al., Cell. Signal., 41:89-96, 2018.
Circu et al., PLOS ONE, 11:e0146931, 2016.

### DiNunzio et al., Mol Pharm., 5(6):968-80, 2008.

### DiNunzio et al., Drug Development and Industrial Pharmacy, 36(9):1064-78, 2010d.

### DiNunzio et al., European Journal of Pharmaceutical Sciences, 40(3):179-87, 2010c.

### DiNunzio et al., European Journal of Pharmaceutics and Biopharmaceutics, 74(2):340-51, 2010a.

### DiNunzio et al., Journal of Pharmaceutical Sciences, 99(3):1239-53, 2010b.

Dunay et al., Antimicrobial agents and chemotherapy; 48(12):4848-54, 2004.

### Engstrom et al., Pharm Res.; 25(6):1334-46, 2008.

Fang et al., Lancet Respir. Med., 2020.
Fonseca et al., J. Biol. Chem., 287:17530-17545, 2012.
Friesen et al., Molecular pharmaceutics, 5(6):1003-19, 2008.
Gassen et al., Nat. Commun., 10:1-16, 2019.
Gurwitz, D. Drug Dev. Res., 2020.

### Handbook of Pharmaceutical Salts: Properties, and Use (P. H. Stahl & C. G. Wermuth eds., Verlag Helvetica Chimica Acta, 2002).

### Hoffmann et al., Cell, 2020.

Ippolito et al., PloS One, 11:e0159675, 2016.

### Jeon et al., 2020.

### Jurgeit et al., PLoS Pathog., 8, 2012.

### Kim and Ryan, Current Treatment Options in Oncology, 13(2):189-200, 2012.

### Lang et al., Mol Pharm., 11(1):186-96, 2014b.

### Lang et al., J Drug Deliv Sci Tec., 24(2):205-11, 2014a.

Li et al., Cancer research, 73(2):483-9, 2013.
Li et al., Cancer letters, 349(1):8-14, 2014.
Liu et al., European journal of pharmaceutics and biopharmaceutics, 96:132-42, 2015.

### Liu et al., Oncotarget, 7(22):32210. 2016.

### Liu et al., Clinical Cancer Research., 2014.

### Liu et al., The Prostate, 75(13):1341-53. 2015.

### Luedeker et al., Crystal Growth & Design, 16(6):3087-3100, 2016.

### Matsubara et al., Clinical Genitourinary Cancer, 16(2):142-8, 2018.

Mook et al., Bioorg. Med. Chem., 23:5829-5838, 2015.
Mostaghel et al., Clinical cancer research, 2011.

### O'Donnell et al., Drug Dev Ind Pharm., 39(2):205-17, 2013.

### Overhoff et al., Pharm Res., 25(1):167-75, 2008.

Overhoff et al., European journal of pharmaceutics and biopharmaceutics, 65(1):57-67, 2007b.

### Overhoff et al., Int J Pharmaceut., 336(1):122-32, 2007a.

### Overhoff et al., J Drug Del Sci Tech., 19(2):89-98, 2009.

Peeri et al., Int. J. Epidemiol., 2020.

### Phillips et al., J Pharm Pharmacol., 64(11):1549-59, 2012.

### Purvis et al., Aaps Pharmscitech., 8(3):E58, 2007.

### Rothan & Byrareddy, J. Autoimmun., 102433, 2020.

Sanphui et al., Cryst. Growth Des., 12(9):4588-4599, 2012.

### Savjani et al., Pharmaceutics, 195727, 2012.

Schweizer et al., PloS one, 13(6):e0198389, 2018.
Sinswat et al., European journal of pharmaceutics and biopharmaceutics, 69(3):1057-66, 2008.

### Takabe et al., Pharmaceutics, 10(2):60, 2018.

Tam et al., Nat. Commun., 9:1-11. 2018.

### Thakkar et al., Hum Vaccin Immunother, 13(4):936-46, 2017.

Tharmalingam et al., Sci. Rep., 8:3701, 2018.
Tortoric et al., Nat. Struct. Mol. Biol., 26:481-489, 2019.
Vincent et al., Virol. J., 2:69, 2005.

### Walls et al., Cell, 2020.

### Wang et al., Genes Dev., 32:1105-1140, 2018.

### Wang et al., Aaps Pharmscitech., 15(4):981-93, 2014.

### Wang et al., Cell Res., 30:269-271, 2020.

### Watts et al., Int J Pharmaceut., 384(1-2):46-52, 2010.

### Watts, et al., 2013.

Wen et al., J. Med. Chem., 50:4087-4095, 2007.
WHO Director-General's opening remarks at the Mission briefing on COVID-19-12 March 2020. www.who.int/dg/speeches/detail/who-director-general-s-opening-remarks-at-the-mission-briefing-on-covid-19---12-march-2020.
Wu et al., J. Antimicrob. Agents Chemother., 48, 2693-2696, 2004.
Xu et al., ACS Infect. Dis., 2020.

### Yan et al., Science, 2020.

### Yang et al., 2010.

### Yang et al., Int J Pharmaceut, 361(1-2):177-88, 2008.

Zhang et al., PloS one; 6(11):e27970, 2011.
Zhang et al., European journal of pharmaceutics and biopharmaceutics, 82(3):534-44, 2012.

1. A delayed release niclosamide composition comprising:
(A) a core comprising:
(i) niclosamide;
(ii) a pharmaceutically acceptable polymer;
(iii) an amorphous solid dispersion (ASD) excipient;
wherein the niclosamide, the pharmaceutically acceptable polymer, and the ASD excipient form an amorphous solid dispersion; and
(iv) a first core excipient; and
(B) a delayed release coating comprising a delayed release polymer;
wherein the core is encapsulated within the delayed release coating.

2. The composition of claim 1, wherein the composition is a tablet.

3. (canceled)

4. The composition of either claim 1, wherein the delayed release coating substantially or entirely encapsulates the core.

5-6. (canceled)

7. The composition according to claim 1, wherein the pharmaceutically acceptable polymer is a vinylpyrrolidone copolymer; 1-vinyl-2-pyrrolidone; and vinyl acetate copolymer; or copovidone.

8-9. (canceled)

10. The composition according to claim 1, wherein the composition further comprises a sealing coating selected from the group consisting of polyvinyl alcohol and a cellulose polymer.

11-12. (canceled)

13. The composition of claim 1, wherein the delayed release coating is a cellulose polymer or an acrylate copolymer.

14. The composition of claim 13, wherein the delayed release coating is a cellulose polymer; ethyl cellulose; hypromellose phthalate; or hypromellose acetate succinate.

15-16. (canceled)

17. The composition according to claim 10 wherein the sealing coating substantially or entirely encapsulates the core.

18-19. (canceled)

20. The composition according to claim 10, wherein the delayed release coating encapsulates the sealing coating.

21-30. (canceled)

31. The composition according to claim 1, wherein the ASD excipient is a vitamin or vitamin derivative selected from the group consisting of a pegylated version of a vitamin; a vitamin E derivative; or D-α-tocopheryl polyethylene glycol succinate.

32-44. (canceled)

42. The composition according to claim 1, wherein the core further comprises a second core excipient.

43. The composition of claim 42, wherein the second core excipient is a modified cellulose derivative.

44. The composition of either claim 42 or claim 43, wherein the modified cellulose derivative is carboxymethyl cellulose.

45-59. (canceled)

60. The composition according to claim 1, wherein the core further comprises a third core excipient.

61. The composition of claim 60, wherein the third core excipient is a salt.

62-63. (canceled)

64. The composition according to claim 1, wherein the core further comprises a fourth core excipient.

65. The composition of claim 64, wherein the fourth core excipient is a lubricant.

66-67. (canceled)

68. The composition according to claim 65, wherein the lubricant is a fatty acid, a salt of a fatty acid, sodium lauryl sulfate, magnesium stearate, magnesium silicate, calcium stearate, sodium lauryl sulphate, sodium stearyl fumarate, magnesium lauryl sulphate, stearic acid, calcium stearate, glyceryl behenate, behenoyl polyoxylglycerides, glyceryl dibehenate, lauric acid, glyceryl monostearate, glyceryl tristearate, myristic acid, palmitic acid, poloxamer, polyethylene glycol, polyethylene glycol 3350, polyoxyl 10 oleyl ether, polyoxyl 15 hydroxystearate, polysorbate 40, polyoxyl 20 cetostearyl ether, polyoxyl 40 stereate, potassium benzoate, sodium benzoate, sorbitan monolaurate, sorbitan monooleate, sodium stearate, sorbitan monopalmitate, sorbitan monostearate, zinc stearate, sorbitan sesquioleate, sorbitan trioleate, or talc.

69-73. (canceled)

74. The composition according to claim 1, wherein the core further comprises a fifth core excipient.

75-135. (canceled)

136. A method of preparing a composition according to claim 1, comprising:
(A) preparing an extrudate comprising niclosamide and a pharmaceutically acceptable polymer to obtain an amorphous solid dispersion;
(B) admixing one or more core excipients to the amorphous solid dispersion to obtain a precursor core;
(C) pressing the precursor core to obtain a core;
(D) coating the core with a clear coating to obtain a clear coated core; and
(E) coating the clear coated core with an acrylate copolymer to obtain a composition.

137-182. (canceled)
